(19) United States
(12) Patent Application Publication
(10) Pub. No.: US 2006/0008906 A1
(43) Pub. Date: Jan. 12, 2006

(54) Electrofusionof cells and apparatus therefore

(76) Inventors: Ivan Nicholas Wills, New South Wales (AU); David Robert James Monaghan, New South Wales (AU)

Correspondence Address: SCULLY SCOTT MURPHY & PRESSER, PC, 400 GARDEN CITY PLAZA, SUITE 300, GARDEN CITY, NY, 11530, US

(21) Appl. No.: 10/516,087

(22) PCT Filed: May 30, 2003
(86) PCT No.: PCT/AU03/00660

§ 371 Date: Jul. 21, 2005

Related U.S. Application Data
(60) Provisional application No. 60/384,882, filed on May 31, 2002.
(60) Provisional application No. 60/387,284, filed on Jun. 7, 2002.

(30) Foreign Application Priority Data
Jun. 19, 2002 (AU) PS 3078
Sep. 5, 2002 (AU) 2002951223
Sep. 5, 2002 (AU) 2002951222

(51) Int. Cl. C12N 15/07; C12N 15/08

(52) U.S. Cl. 435/451

(57) ABSTRACT

The present invention relates to a method and apparatus for fusing first and second cells. In particular, the method includes positioning the first and second cells between two electrodes (35) in a fluid filled container (40) using a pipette system (33), with the first and second cells being held separated from each electrode. Once this has been achieved a current having a predetermined waveform is applied to the electrodes (35) to generate a predetermined electric field thereby causing the cells to fuse.

### BACKGROUND OF THE INVENTION

The present invention relates to a method and apparatus for fusing first and second cells, and in particular, for producing hybrid cells by electrofusion.

### DESCRIPTION OF THE PRIOR ART

The reference to any prior art in this specification is not, and should not be taken as, an acknowledgement or any form of suggestion that the prior art forms part of the common general knowledge in Australia.

Previously it has be known to fuse cells together using a variety of techniques such as chemical fusion employing polyethylene glycol, biological methods such as viruses or viral proteins or electrofusion of cells in suspension. Methods of fusion carried out by chemical or biological means often suffer from problems associated with contamination, low efficiency and cytotoxity.

There are a number of advantages to using electrofusion for producing hybrid cells. The fusion conditions can be better controlled and optimised depending on the type of cell to be fused than chemical or biological fusion allows. This allows electrofusion to lead to an increase in cell fusion efficiency.

The basis for electrofusion is to expose pairs of cells, in close membrane contact, to an electric field that induces a sufficient voltage across their cellular membrane to cause mechanical breakdown of the cell membrane and the formation of pores at the point of cell-to-cell contact. Ideally the pores should be of sufficient size to allow transfer of cellular material between the two cells, particularly allowing the two nuclei to come together and subsequently fuse. This formation of pores should also be reversible such that any pores formed at points other than that of cell-to-cell contact seal quickly.

A further process known as dielectropherisis (DEP) in which a non-uniform alternating electric field is applied to the cells in order to ensure good cellular contact prior to fusion. Typically DEP is employed to form ‘pearl chains’ of large numbers of cells between the electrodes in the fusion container.

Whilst electrofusion has a number of advantages over chemical and biological processes, all of these methods fuse cells simultaneously in large numbers. The pairing of the cells, necessary to form hybrid cells, is therefore completely random. Accordingly, the current methods are limited in that they require a chemically sensitive immortal cell such that unfused and self-fused cells can be eliminated from the final culture.

Furthermore, these ‘bulk’ methods also do not lend themselves to hybrid creation from rare cells. Typically fusion methods require millions of cells in order to overcome the problems associated with ensuring cellular contact between the desired cells. In some cases the number of target cells for fusion might only number in 10's-100's. Further, in the recovery phase, whereby cells from the fusion process are plated out and the chemical selection process to remove unfused cells takes place, there is no guarantee of clonal purity in the final product. This plating of cells is also extremely time consuming.

It would therefore be desirable to provide a method whereby lesser numbers of cells could be selected, electrically fused and recovered to grow.

An example of a system suitable for performing electrofusion on a small number of cells is described in WO93/05166. This describes apparatus that utilises an electrode coated with ligands. In use, the ligands are used to attract target cells bearing complimentary ligands. Once the ligands are bound, the cells are therefore effectively bound to the electrode. Accordingly, at this point the target cells can be brought into contact with partner cells allowing the cells to be fused.

However a number of drawbacks exist with these techniques. Firstly, the target cell is held in contact with the electrode during the electrofusion process. As a result the cell is usually subject to an intense electric field which tends to damage the cell. Secondly, the technique can only be performed with a number of target cells attached to the electrodes, and a number of partner cells. Accordingly, this means that any cells successfully fused may be separated out from cells that do not fuse, which can be a complex procedure. A further disadvantage of this technique is that cells can bind to the electrode non-specifically leading to false fusion events taking place.

A second example of a system for performing cell fusion on individual cells is described in WO01/09297. In this example, cells are manipulated using a combination of optical trapping, and pushing the cells with micro-electrodes. Once the cells are correctly positioned relative to each other, an electric field is applied to the cells to cause the cells to fuse.

However, a number of significant drawbacks exist with the apparatus. Firstly, the presence of the laser and associated optics required to manipulate the cells results in the apparatus being expensive, time consuming to configure and complicated to use. Secondly, the electrodes must be significantly smaller than the cells in size, to allow manipulation of the cells by pushing. As a result, the electrodes are again expensive, difficult to construct and extremely fragile, thereby further increasing the cost and complexity of the apparatus.

In addition to this, touching the cells with the electrodes can lead to additional problems, such as burning of the cells. Even in the event that a signal is not being applied to the electrodes when the cell is pushed, the electrodes can retain a residual field from when they are last used. In this case, contact of the cell with the electrode can cause the field to be discharged, thereby damaging the cell.

Finally, the use of the laser trapping and electrodes to manipulate cells is difficult to achieve manually as described in WO01/09297. This not only means that training is required to perform cell fusion using the apparatus, but also means the cell fusion process itself can be time consuming.

### SUMMARY OF THE PRESENT INVENTION

In a first broad form the present invention provides a method of fusing first and second cells, the method including:
a) Selecting the first and second cells;
b) Positioning the first and second cells between two electrodes in a fluid filled fusing container, the first and second cells being held in suspension separated from each electrode; and,
c) Applying a current having a predetermined waveform to the electrodes to cause the cells to fuse.

Typically the cells are held in suspension between the electrodes.

The method typically includes generating a DEP field, the DEP field being adapted to urge the cells towards each other.

The predetermined waveform may include a current representing the DEP field. Alternatively, the method can include applying the DEP to a pair of second electrodes.

The method generally includes:
a) Applying a DEP current to the pair of second electrodes;
b) Positioning the first cell in the fusing container, the alternating field acting to attract the first cell towards one of the second pair of electrodes; and,
c) Positioning the second cell in the fusing container, the alternating field acting to attract the second cell towards the first cell.

At least one of the first and second cells is generally positioned in contact with at least one of the second pair of electrodes.

The method of selecting the first and second cells typically includes using a pipette to extract:
a) The first cell from a group of first cells held in a first container; and,
b) The second cell from a group of second cells held in a second container.

The method of positioning the first and second cells between the two electrodes usually includes:
a) Using the pipette to position the first cell in the fusing container;
b) Using the pipette to position the second cell in the fusing container, adjacent the first cell;
c) Positioning the electrodes such that the first and second cells are located substantially between the electrodes.

The pipette is typically coupled to:
a) A drive system adapted to move the pipette with respect to the first, second and fusing containers; and,
b) An actuator adapted to actuate the pipette to thereby expel or draw in fluid through a port.

In this case, the method usually includes using a controller coupled to the drive system and the actuator to move and actuate the pipette.

The method of selecting a cell preferably includes causing the controller to:
a) Move the pipette such that the port is adjacent a cell having predetermined characteristics, the cell being held in fluid suspension in the respective container;
b) Actuate the pipette to draw in fluid through the port, thereby drawing in the cell and the surrounding fluid.

The method of using the pipette to position the second cell adjacent the first cell generally includes causing the controller to:
a) Move the pipette such that the port is adjacent the first cell in the fusing container;
b) Cause the pipette to expel fluid through the port, thereby expelling the second into the fluid in the fusing container;
c) Move the pipette such that the port is as close as possible to both the first and second cells;
d) Cause the pipette to draw in fluid through the port, thereby drawing in the first and second cells and the surrounding fluid;
e) Cause the pipette to expelling the first and second cells into the fluid in the fusing container; and,
f) Repeat steps (c) to (e) until the first and second cells are within a predetermined distance.

The electrodes may be coupled to an electrode drive system adapted to move the electrodes with respect to the fusing containers, in which case the method typically includes using a controller coupled to the electrode drive system to position the electrodes in the fusing chamber.

The electrodes may be coupled to a signal generator, in which case the method of applying the alternating current includes causing the signal generator to apply a predetermined waveform to the electrodes.

If the first and second cells having a respective cell type, the method preferably includes using a controller coupled to the signal generator to select the current in accordance with the cell types of the first and second cells.

The fist and second cells may be the same type of cell, the first and second group of cells being the same group.

In a second broad form the present invention provides apparatus for fusing first and second cells, the apparatus including:
a) A fluid filled fusing container,
b) At least two electrodes adapted to be positioned in the fusing container in use;
c) A selector for:
   i) Selecting a first cell from a group of first cells held in a respective container; and,
   ii) Selecting a second cell from a group of second cells held in a respective container;
   iii) Positioning the first and second cells in the fusing container, the first and second cells being held in suspension; and,
d) A signal generator coupled to the electrodes, the signal generator being adapted to cause a field having a predetermined waveform to be generated between the electrodes, thereby causing the cells to fuse.

The selector is preferably a pipette.

The apparatus generally further includes:
a) A drive system adapted to move the pipette with respect to the first, second and fusing containers; and,
b) An actuator adapted to cause the pipette to expel or draw in fluid through a port.

The electrodes may be coupled to the fusing container.

Alternatively the apparatus can include an electrode drive system adapted to move the electrodes with respect to the fusing containers.

The current waveform typically includes a fusion pulse, the signal generator being adapted to apply the fusion pulse to the electrodes to generate an electric field pulse thereby causing the cells to fuse.

The current waveform preferably also includes a DEP current, the signal generator being adapted to apply the DEP current to the electrodes to generate a DEP field thereby urging the cells towards each other.

The apparatus may include a pair of second electrodes, the pair of second electrodes being coupled to a second signal generator, the second signal generator being adapted to generate a DEP current, the DEP current being applied to the pair of second electrodes to generate a DEP field thereby urging the cells towards each other.

In this case, the pair of second electrodes being provided on the fusing container surface.

The apparatus also typically includes a controller adapted to control the fusing of the cells by controlling operation of at least one of:
a) The pipette;
b) The electrodes; and,
c) The signal generator.

The controller typically includes a processor coupled to at least one of:
a) The drive system and the actuator, the processor being adapted to move and actuate the pipette;
b) The electrode drive system, the processor being adapted to move the electrodes; and,
c) The signal generator, the processor being adapted to cause the signal generator to generate the field having the predetermined waveform.

The controller may include a detector adapted to detect the position of cells within the containers, in which case the processor can be responsive to the detector to move at least one of the electrodes and the pipette in response to the position of detected cells.

Alternatively, or additionally, the processing system may include an input for receiving input commands from a user.

The processor can be coupled to a store for storing waveform data representing a number of different predetermined waveforms, the processor being adapted to select one of the number of predetermined waveforms in response to the input commands received from the user.

The processor can also being adapted to move at least one of the electrodes and the pipette in response to the input commands received from the user.

Typically the controller is adapted to cause the cells to fuse by causing the apparatus to perform the method of the first broad form of the invention.

In a third broad form the present invention provides, a controller for controlling apparatus for fusing first and second cells, the apparatus including:
a) A fluid filled fusing container;
b) At least two electrodes;
c) A selector;
d) A signal generator coupled to the electrodes;
Wherein, in use, the controller is adapted to cause the cells to fuse by:
   i) Causing the selector to:
      (1) Select a first cell from a group of first cells held in a respective container; and,
      (2) Select a second cell from a group of second cells held in a respective container; and,
      (3) Position the first and second cells in the fusing container between the electrodes, the first and second cells being held in suspension;
   ii) Positioning the electrodes in the fusing container; and,
   iii) Causing the signal generator apply a field having a predetermined waveform to the electrodes, thereby causing the cells to fuse.

The controller can also be adapted to position the cells in the fusing container.

In this case, the controller typically includes a processor coupled to at least one of:
a) A drive system adapted to move the pipette with respect to the first, second and fusing containers;
b) An actuator adapted to cause the pipette to expel or draw in fluid through a port;
c) An electrode drive system adapted to move the electrodes with respect to the fusing containers; and,
d) The signal generator.

The current waveform typically includes a fusion pulse, the controller being adapted to cause the signal generator to apply the fusion pulse to the electrodes to generate an electric field pulse thereby causing the cells to fuse.

The current waveform usually includes a DEP current, the controller being adapted to cause the signal generator to apply the DEP current to the electrodes to generate a DEP field thereby urging the cells towards each other.

The apparatus can include a pair of second electrodes, the pair of second electrodes being coupled to a second signal generator, the controller being adapted to cause the second signal generator to generate a DEP current, the DEP current being applied to the pair of second electrodes to generate a DEP field thereby urging the cells towards each other.

The controller is typically adapted to operate for use with apparatus of the second broad form of the invention.

In this case, the controller is preferably adapted to cause the apparatus to perform the method of the first broad form of the invention.

In a fourth broad form the present invention provides a computer program product for controlling apparatus for fusing first and second cells, the computer program product including computer executable code which when executed by a suitable processing system causes the processing system to operate as the controller of the third broad form of the present invention.

In a fifth broad form the present invention provides a pipette system for manipulating particles, the pipette system including:
a) A nozzle for containing fluid in use, the nozzle including a port;
b) An actuator coupled to the nozzle, the actuator being adapted to draw in and/or expel fluid through the port; and,
c) An electrode coupled to the nozzle adjacent the port, the electrode being adapted to cooperate with a second electrode to allow an electric field to be applied to coupled to one or more particles positioned adjacent the port.

The electrode is usually formed a conductive tube.

The electrode may be formed from a stainless steel tube having a diameter of approximately 10 mm.

The pipette system can include a drive system adapted to move the pipette system to be with respect to a fluid filled container to thereby allow particles to be positioned in or removed from fluid in the container.

The pipette system can include a signal generator coupled to the electrode for generating a predetermined electric field between the electrode and a second electrode positioned in the container.

The pipette system typically includes a controller adapted to control the drive system, the actuator and the signal generator to thereby apply an electric field to a particle by:
a) Positioning the particle in the container adjacent the second electrode using the pipette;
b) Positioning the pipette port adjacent the particle in the container; and,
c) Activating the signal generator.

The controller is typically adapted to fuse cells, by:
a) Positioning a first cell in the container adjacent the second electrode using the pipette;
b) Positioning a second cell in the container adjacent the first cell using the pipette;
c) Positioning the pipette port adjacent the first and second cells, such that first and second cells are substantially between the electrodes; and,
d) Activating the signal generator to cause a predetermined field sequence to be applied to the cells, thereby causing the cells to fuse.

The pipette system generally further includes:
a) A radiation source; and,
b) A waveguide having a first end coupled to the radiation source and a second end coupled to the nozzle adjacent the port to thereby allow radiation from the radiation source to impinge on particles positioned adjacent to the port in use.

The pipette system can include a detector, the detector being adapted to detect radiation emitted by the particle.

The detector can be coupled to the first end of the waveguide, to thereby detect radiation emitted from the particle.

The radiation is typically a laser, although other sources, such as LEDs may be used.

The waveguide can be a fibre optic cable, or alternatively can being formed from the nozzle, the nozzle including a shaped portion to allow the radiation from the radiation source to enter the nozzle and pass along at least a portion of the nozzle, the radiation being emitted from the nozzle through the port.

The pipette system generally includes a controller adapted to perform at least one of:
a) Activating the actuator to thereby cause fluid to be drawn in and/or expelled through the port; and,
b) Activating the radiation source, to thereby expose a particle to radiation.

The drive system can be coupled to a controller, the controller being adapted to recover particles having predetermined properties from the container by:
a) Positioning the pipette system such that the port is adjacent to a particle;
b) Activating the radiation source to thereby expose the particle to radiation;
c) Detect any radiation emitted by the particle;
d) Determine if the particle has the predetermined properties in accordance with the detected radiation; and,
e) In accordance with a successful comparison, activate the actuator to thereby draw fluid into the nozzle through the port, thereby recovering the particle.

The actuator can include:
a) A fluid reservoir;
b) A flexible tube coupling the nozzle to the fluid reservoir;
c) An arm positioned so as to partially compress the tube;
d) An actuator drive system adapted to move the arm so as to perform at least one of:
   i) Further compressing the tube to thereby expel fluid from the port; and,
   ii) Decompressing the tube to thereby draw fluid in through the port.

The actuator drive system generally includes:
a) A first actuator drive for moving the arm with respect to the tube; and,
b) A second actuator drive formed from an arm end portion, the arm end portion being in contact with the tube in use, the second actuator drive being adapted to cause the arm end portion to expand or contract.

The pipette system usually includes a controller coupled to the actuator drive system, the controller being adapted to operate the actuator drive system to thereby draw fluid in or expel fluid through the port.

The drive system can be coupled to the controller, the controller being adapted to recover particles from the fluid by:
a) Positioning the pipette system such that the port is adjacent to a particle; and,
b) Activate the actuator drive system to thereby draw fluid into the nozzle through the port, thereby recovering the particle.

The tube can be formed from silicon tubing.

### BRIEF DESCRIPTION OF THE DRAWINGS

An example of the present invention will now be described with reference to the accompanying drawings, in which:

FIG. 1 is a block diagram of an example of apparatus for fusing cells;

FIG. 2 is a schematic diagram of the apparatus of FIG. 1;

FIG. 3 is a schematic diagram of the pipette of FIG. 1;

FIG. 4 is a flow chart of an overview of the process of fusing cells using the apparatus of FIG. 1;

FIGS. 5A to 5C are a flow chart of the process of fusing cells implemented by the apparatus of FIG. 1;

FIGS. 6A and 6B are schematic diagrams of cells being drawn into and expelled from the pipette of FIG. 3;

FIGS. 6C and 6D are schematic diagrams of the arrangement of the electrodes and cells in the fusion well during operation of the apparatus of FIG. 1; FIGS. 7A to 7G are examples of pulse sequences that may be used in the apparatus of FIG. 1;

FIG. 8A is a schematic plan view of a second example of apparatus for fusing cells;

FIG. 8B is a schematic side view of the modified well array of FIG. 8A;

FIG. 9A is a schematic plan view of a third example of apparatus for fusing cells;

FIG. 9B is a schematic side view of one of the cells shown in FIG. 9A;

FIG. 9C is a schematic perspective view of the first electrodes of FIG. 9A;

FIG. 10 is a schematic diagram of the pipette of FIG. 3 modified to include an electrode;

FIG. 11 is a block diagram of a modified version of the apparatus of FIG. 1 adapted to use two of the pipettes shown in FIG. 10;

FIG. 12 is a schematic diagram of the apparatus of FIG. 11;

FIG. 13A is a schematic diagram of the pipette of FIG. 10 modified to include a radiation source;

FIG. 13B is a schematic diagram of the pipette of FIG. 3 modified to include an alternative radiation source;

FIG. 14 is a schematic diagram of the pipette of FIG. 3 retrieving a number of cells;

FIG. 15 is a schematic diagram of the pipettes of FIG. 11 positioning cells for subsequent fusion;

FIG. 16 is a schematic diagram of the pipettes of FIG. 11 and fused cells;

FIG. 17 is a schematic diagram of the pipette of FIG. 3 modified to include a radiation source;

FIG. 18A is a schematic diagram of the pipette of FIG. 3 with an alternative actuator;

FIG. 18B is a schematic diagram of the operation of the actuator of FIG. 18A;

FIG. 18C is a schematic diagram of a first example of the pipette of FIG. 18A modified for use with a bladder;

FIG. 18D is a schematic diagram of a second example of the pipette of FIG. 18A modified for use with a bladder;

FIG. 19 is a schematic diagram of a cutting tool used for cutting cells;

FIG. 20 is a block diagram of an example of apparatus for automatically fusing cells; and,

FIG. 21 is a schematic diagram of the apparatus of FIG. 20.

### DETAILED DESCRIPTION OF THE PREFERRED EMBODIMENTS

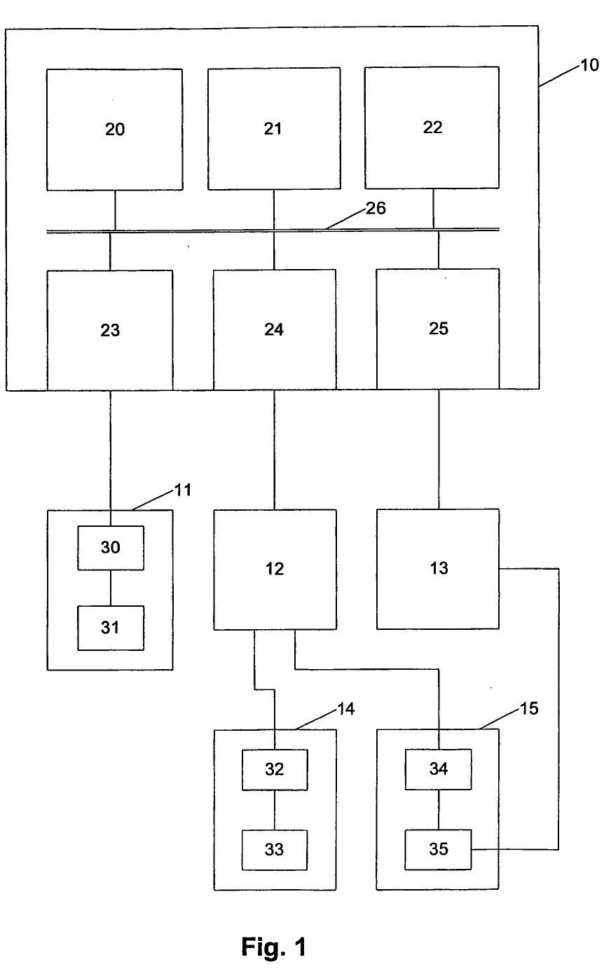

An example for apparatus suitable for implementing the present invention will now be described with reference to FIGS. 1, 2 and 3.

As shown in FIG. 1, the apparatus includes a processing system 10 coupled to an imaging system 11, a control system 12 and a signal generator 13. The control system 12 is coupled to a pipette system 14 and an electrode system 15, as shown.

The processing system 10 includes a processor 20, a memory 21, an input/output (I/O) device 22, an image interface 23, a control interface 24, and a signal interface 25, coupled together via a bus 26. The processing system may therefore be any one of a number of systems, such as a suitably programmed computer, specialised hardware, or the like. In any event, the I/O device typically includes a display, such as a computer monitor or the like, a keyboard, and one or more other input devices such as a mouse, joystick trackball or the like.

The imaging system 11 includes a camera 30 such a CCD camera or the like which is coupled to a microscope 31. The imaging system 11 is connected to the processing system via the image interface 23.

The pipette system 14 includes a pipette shown generally at 33 that is coupled to the control system 12 via a drive system 32. In use, the control system 12 is coupled to the processor via the control interface 24, thereby allowing the drive system 32 to be used to control motion and operation of the pipette, as will be described in more detail below.

Similarly, the electrode system 15 is formed from two electrodes 35 coupled to the control system 12 via a drive system 34. Again, the control system 12 allows the drive system 34 to control the position of the electrodes, as will be described in more detail below.

In use, the system allows a user to select and move individual cells using the pipette system 14. When appropriate cells are placed next to each other, this allows an electric field to be applied to the cells using the electrodes 35 thereby causing the cells to fuse.

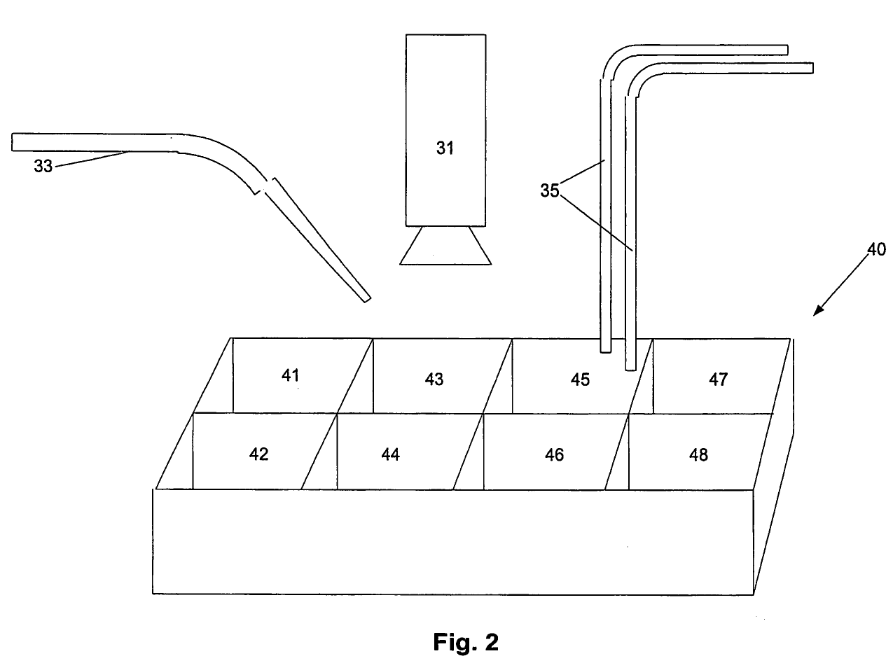

In order to achieve this, the apparatus is arranged as shown schematically in FIG. 2 such that the pipette 33 and the electrodes 35 may be moved relative to a well array shown generally at 40. This allows cells to be moved between the wells 40, 41, 42, 43, 44,45, 46, 47, 48, as shown.

Movement of the pipette and the electrodes 35 is achieved by operation of the corresponding drive system 32, 34. Accordingly, it will be appreciated that the processing system 10 may be used to control positioning of the pipette 33 and the electrodes 35 allowing the pipette 33 and the electrodes 35 to be inserted into and positioned within a respective one of the wells 41, . . . , 48.

Furthermore, the microscope 31 is arranged to image selected wells 41, . . . , 48 such that the representation of the contents of a selected well can be displayed to the user using the I/O device 22.

In general, the processing system 10 is adapted to control the pipette 33 and the electrodes 35 in accordance with input commands received from the user via the I/O device 22. In order to achieve this, the processing system 10 must be able to perform a number of functions simultaneously, such as:
Presenting an image of the well array 40 to the user on the I/O device 22;
Responding to commands input via the I/O device 22 to move and, if required, actuate the pipette system 14;
Responding to command inputs via the I/O device 22 to move the electrodes 35; and,
Responding to commands input via the I/O device 22 to apply an electrical signal to the electrodes 35.

This is achieved by having the processor 20 execute appropriate application software which is stored in the memory 21.

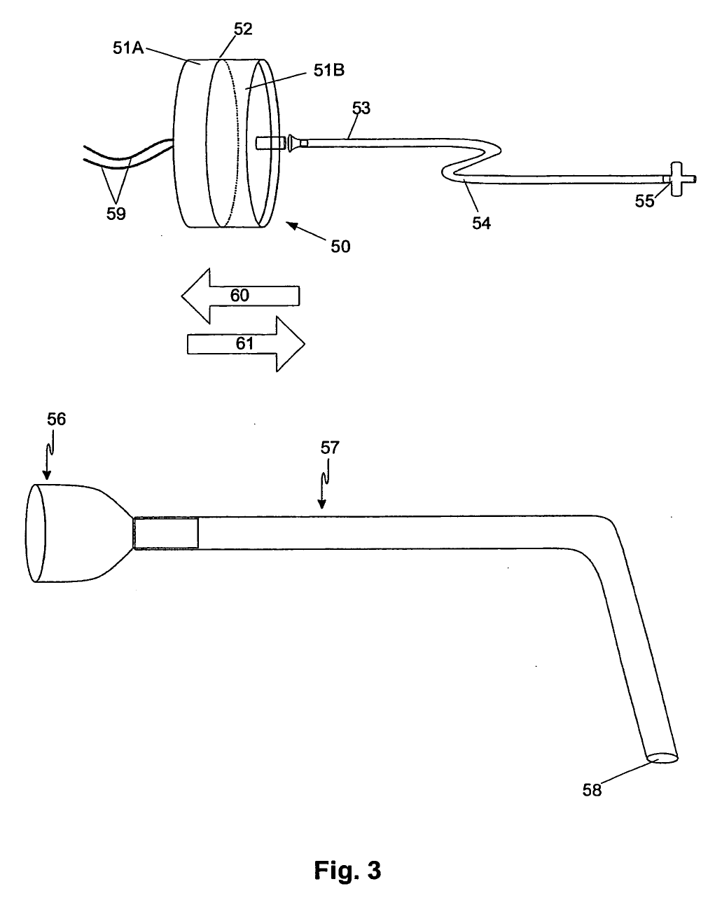

The pipette is shown in more detail in FIG. 3. As shown, the pipette 33 is formed from a housing 50 defining a chamber that is divided into two portions 51A, 51B by a piezoelectric element 52, as shown. The chamber 51B is coupled by a port 53 to a flexible tube 54. The flexible tube 54 includes a male coupling 55 that is adapted to cooperate with a female coupling 56 positioned on a shaped glass nozzle 57 having an aperture 58, as shown.

In use, the chamber 51B, the port 53, the flexible tube 54 and the glass nozzle 57 are filled with fluid, with the chamber 51A being filled with air and sealed. Applying a current to the piezo-electric element 52, via leads 59, causes the element to move, with the direction of movement depending on the polarity of the applied current.

Thus, in use, with the aperture 58 positioned in fluid in one of the wells 41, . . . , 48, causing the piezo-electric element 52 to move in the direction of the arrow 60 will increase the volume of the chamber 51B, thereby causing fluid to be drawn through the aperture 59. Similarly, causing the piezo-electric element 52 to move in the direction of arrow 61 will decrease the volume of the chamber 51B, thereby causing fluid to be expelled through the aperture 58.

Accordingly, the pipette can be activated to draw in or expel fluid through the aperture 58 depending on the polarity of the current applied to the leads 56. Accordingly, in use, the leads 56 are coupled to either the drive system 32, or a separate activation system, to allow a suitable current to activate the pipette as required.

The manner in which the apparatus is used to fuse cells will now be described.

Overview

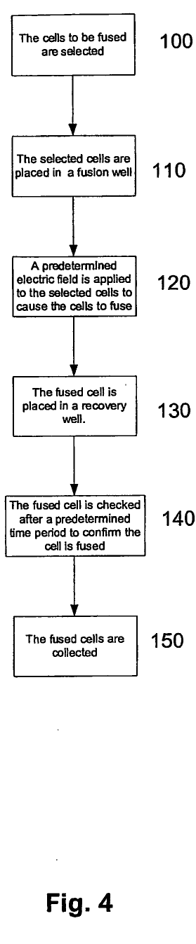

An overview of the method of fusing cells in accordance with the present invention will now be described with reference to FIG. 4.

In particular, at step 100, the user selects the cells to be fused. At step 110, the cells are placed in a fusion well.

At step 120 a predetermined electric field is applied to the selected cells to cause the cells to fuse.

Cells that are placed in an electric field will distort the field in their immediate vicinity. The field distortion is dependent on the geometry and electrical properties of the particle and that of the surrounding particles. Living cells have interior (cytoplasm) that is highly conductive, due to the accumulation of ions such as potassium (K+) ions, and a relatively high dielectric constant. The membrane surrounding has a very low conductivity and a lower dielectric constant.

Accordingly, the degree of the distortion of the field both inside and outside of the cell is a very strong function of the frequency of the applied electric field. As a result when placed in a non-uniform electric field cells will experience a force whose magnitude and direction will vary in a complicated manner with the frequency of the applied field. This effect can be exploited to selectively manipulate living cells using radio-frequency alternating electric fields created via suitable electrodes. The movement of particles in AC electric fields is referred to as ‘dielectropherisis’ (DEP) and is independent of any net charge on the particle.

The application of the radio-frequency electric fields, typically in the region 10-10,000 kHz, exerts a positive DEP force on the two cells, urging the cells into close contact with each other. A stronger electric field is then used in order to induce electrical breakdown of each cell's membranes at their point of contact. This controlled electro-poration triggers a process of cell fusion that is somewhat akin to reverse-mitosis. This in turn creates a fused hybrid cell that has a genetic makeup that is a combination of the two original cells that were fused.

The fused cell is then generally placed in a recovery well at step 130 before being checked after a predetermined time period to confirm the cell has fused at step 140.

The fused cells can then be collected at 150 and used as required.

### DETAILED DESCRIPTION

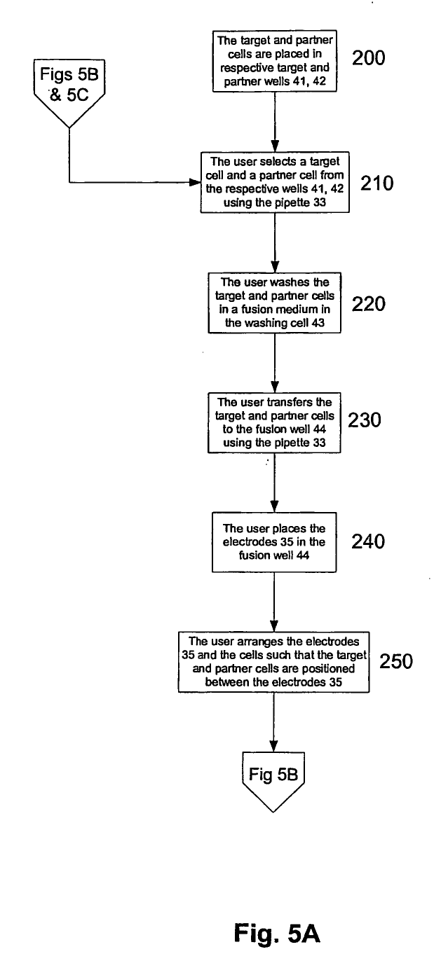

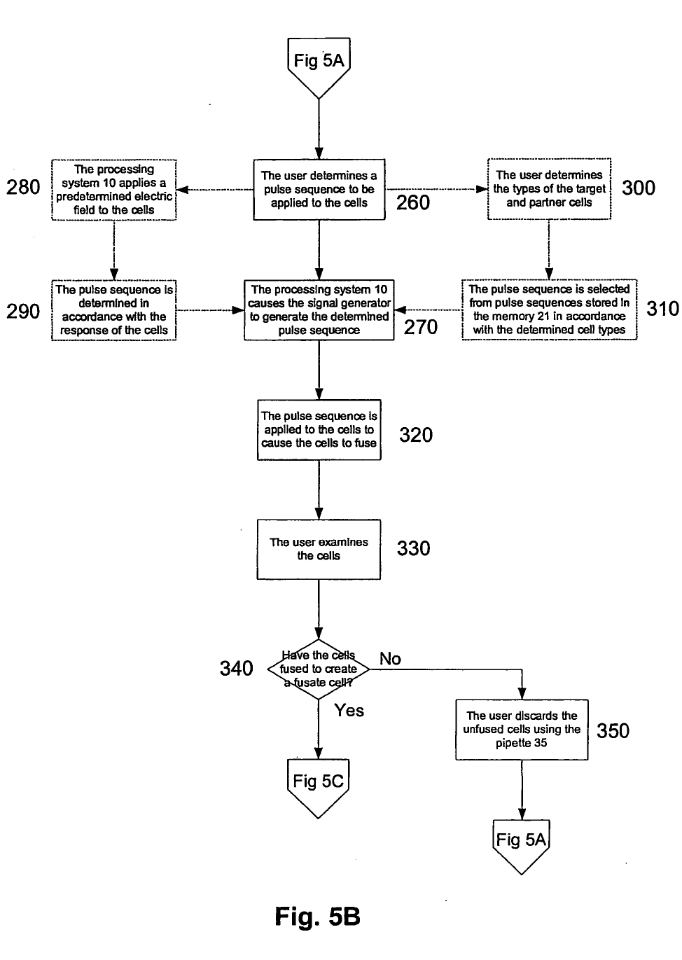

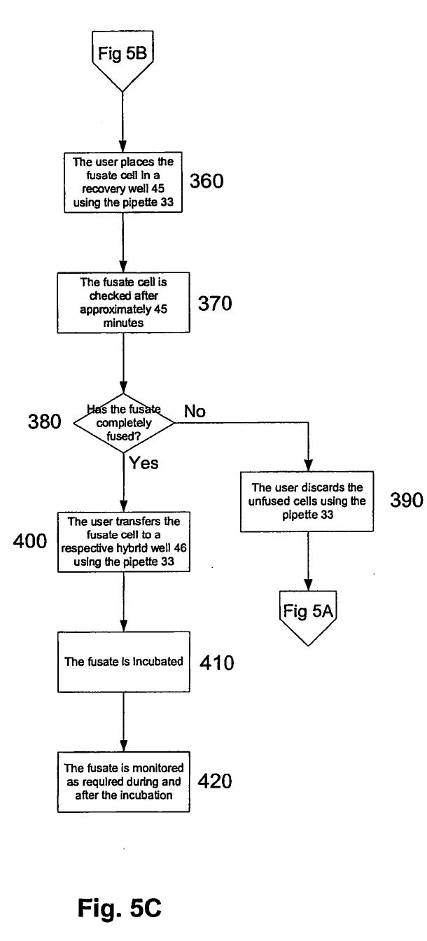

A detailed example of the method of using the apparatus of the present invention will now be described with reference to FIGS. 5A, 5B, 5C and 5D.

In this example, the well array 40 includes a target well 41, a partner well 42, a washing well 43, a fusion well 44, a recovery well 45 and a hybrid well 46 the purpose of which will be described in more detail below.

At step 200 the target and partner cells are placed in respective target and partner wells. This procedure will generally involve suitable preparation of the cells, which may be achieved in a number of manners. Thus, for example, this may require that the cells are recovered from sample plates and washed in appropriate enzyme solutions.

The well array would then be sterilised before appropriate fluids are inserted into the wells to be used. The target and partner cells are then placed in the target and partner wells, 41, 42 respectively, with the cells being held in suspension in respective enzyme solutions.

At step 210, the user selects a target cell from the target well 41 using the pipette 33. In order to achieve this, the user will arrange the well array 40 such that the target well 41 is imaged by the imaging system. Accordingly, the target well 41 is placed under the microscope 31 so that the camera 30 may generate an image signal and transfer this to the image interface 23. The image signal will then generally undergo some pre-processing in the image interface 23 before being transferred to the processor 20 for any subsequent further processing.

Thus, for example, the image interface 23 may be formed from an image capture card, which is used to capture images from incoming image signals. The captured image is then formatted by the processor 20 before being presented to the user using the I/O device 22.

The user adjusts the relative position of the microscope 31 and the well array until a suitable target cell is shown. The user then uses the processing system 10 to control the position of the pipette 33. In particular, this is usually achieved by having a joystick I/O device 22, with the processor 20 responding to signals from the joystick to generate commands which are transferred via the control interface 21 to the control system 12. The control system will typically be formed from a motion control addressing amplifier, which is coupled to a drive system 32, such as suitable stepper or DC servo motors.

By use of appropriate sensitivity control, this allows the position of the pipette to be controlled to high degree of accuracy. By arranging the microscope such that the pipette is shown in the image presented on the display, this allows the user to position the pipette 33 with the pipette aperture 58 adjacent the selected cell.

At this point, the user activates the pipette 33 to draw fluid in through the aperture 58. The cell and the surrounding fluid will be drawn into the pipette, allowing the target cell to be removed from the target well 41.

Sometimes, it is difficult to separate individual cells within the wells. This can be overcome by repeatedly operating the pipette to cause the pipette to repeatedly draw in and expel fluid via the pipette aperture 58. Agitation of the fluid medium and repeated movement of the cells through the pipette aperture 58 will usually allow a cell to be separated from surrounding cells.

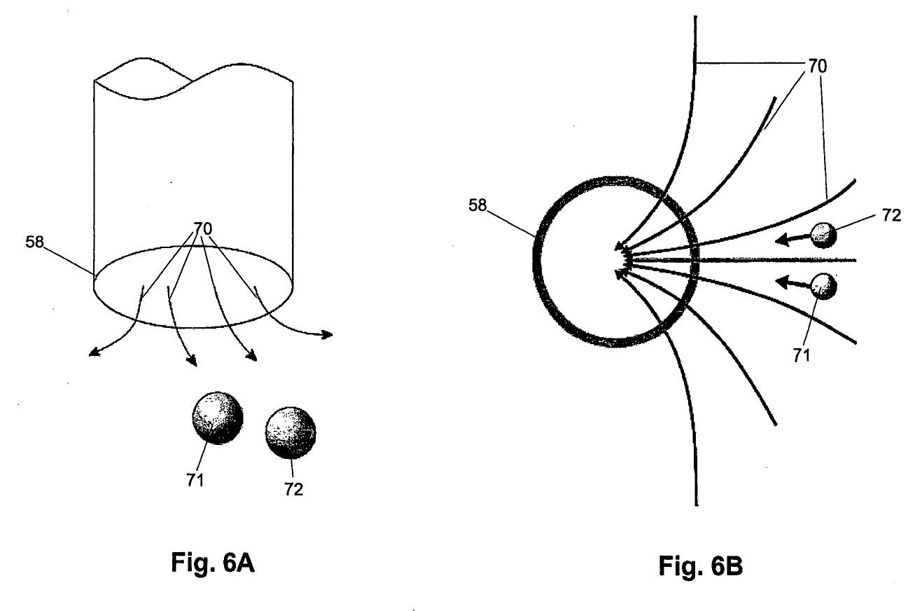

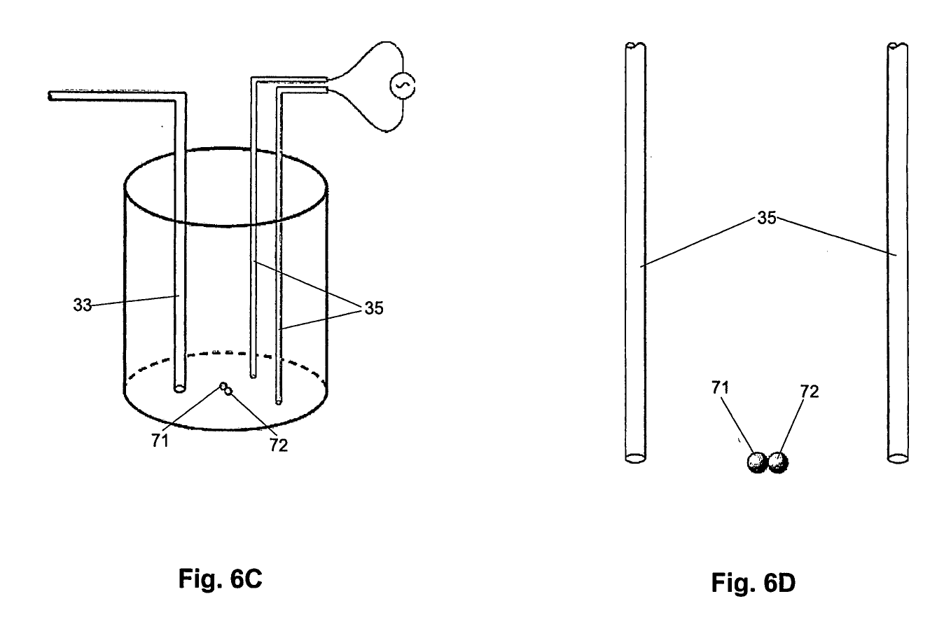

An example of this is shown in FIG. 6A, which shows the hydrodynamic streamlines 70 as fluid is expelled from the pipette aperture 58. As shown, the hydrodynamic stream-lines, which represent lines of constant force, spread out away from the pipette aperture 58. Similarly, as the cells, shown at 71, 72, are entrained in the fluid flow, this will tend to cause the cells 71, 72 to separate as they are expelled away from the pipette aperture 58.

In any event, once the user has selected the target cell at step 210, the user washes the target cell in a fusion medium in the washing well 43. In order to do this, the pipette containing the respective cell is positioned in the washing well 43, using the imaging and control system 11, 12 to move the pipette 33 as described above. Once the pipette 33 is positioned inside the washing well 43, the pipette is repeatedly activated to cause fluid to be drawn in through and expelled through the pipette aperture 58. In this way, the cell is repeatedly placed in the fusion medium in a washing well 43 and then removed. This action causes the cell to be washed.

Furthermore, when the user transfers the target and cell to the fusion well 44 at step 230, this is achieved by positioning the pipette 33 in the washing well 43 and drawing the target and cell into the pipette 33 through the pipette aperture 58. Accordingly, at this point the target cell is surrounded in fusion medium as opposed to in the medium contained in the target well 41.

The user then uses the pipette 33 to place the target cell into the fusion well 44 at step 230. Steps 210 to 230 are repeated for the partner cell, with the partner cell being placed in the fusion well 44 next to the target cell at 230.

As an alternative to performing steps 210 to 230 separately for each cell, the target and partner cells may be selected from the respective wells and then washed together in the washing cell 43 being transferred simultaneously to the fusion well 44.

As will be described in more detail below, it is preferable for the cells 71, 72 to be positioned adjacent to each other. In order to achieve this, it is preferable to first place the target or partner cell 71 in the fusion well 44 and then place the other partner or target cell 72 adjacent thereto.

In general as adding the second cell 72 will cause fluid to be transferred into the fusion well 44, this also causes movement of the first cell 71. It is then generally necessary to repeatedly activate the pipette 33 until the both cells can be drawn in to the pipette simultaneously. As shown in FIG. 6B, when the cells 71, 72 are drawn in to the pipette aperture simultaneously, the hydrodynamic lines of force 70 converge as the fluid enters the aperture 58. Accordingly, this draws the cells 71, 72 together. The cells can then be expelled from the pipette 33 with the cells being sufficiently close for the fusion process to be performed.

In any event, once the user has positioned the target and partner cells in the fusion well at 230 the user then arranges to place the electrodes 35 in the fusion cell 44 at step 240. Again, in order to achieve this, the imaging system 11 is positioned such that the I/O device 22 presents the user with an image of the fusion cell 44.

The user can then alter the position of the electrodes 35 by providing appropriate commands via the I/O device 22. Again, this is usually achieved by having a respective joystick or the like provide control signals to the processor 20. The processor then transfers appropriate command signals via the control interface 24 to the control system 12. The control system then activates the drive system 34, thereby casing the electrodes 35 to move as directed by the user.

An example of the relative positioning of the electrodes 35, the cells 71, 72 and the pipette 33 at this stage is shown in FIGS. 6C and 6D, which show a perspective and end on view of the fusion well 44 prior to fusion being performed. Thus, as shown, the cells 71, 72 are positioned close to each other substantially between the electrodes 35. At this stage the cells need not be in contact as they will in any event be urged together by the applied electrical field as will be described in more detail below.

As shown in FIG. 5B, the next step is for the user to determine the sequence of electric fields that are to be applied to the cells at step 260 before using the processing system 10 and the signal generator 13 to generate the determined pulse sequence at step 270.

The manner in which the user determines the electric field will vary depending on the particular implementation of the invention. A first example by which this may be achieved is shown in steps 280, 290. In this case, the processing system 10 applies a predetermined electric field to the partner and target cells. The response of the cells in the electric field is then used to determine the electrical parameters employed for the DEP electric field (in order to bring the cells together). The response can also be used to determine the fusion pulse sequence (including the frequency and amplitude) required to fuse any particular pair of cells. In particular, the processing system 10 will apply a field having a predetermined frequency. The frequency can then be fine adjusted until an optimum frequency is determined at which the force that attracts the cells to cells move toward each other is optimal for the required conditions. This response of the cells to the DEP electric field will occur due to the generation of electric dipoles within the cells, as described above.

The response of the cells to the electric field can be monitored either automatically by having the processor 20 execute appropriate image recognition software, or manually by the user. The processor would then select a pulse sequence from a number of pulse sequences stored in the memory 32. The pre-programmed pulse sequences would be stored in a look up table (LUT), or the like, in accordance with the field applied to obtain the desired response. It will be appreciated that this information may need to be determined initially. Accordingly, each time a new lineage of target and partner cell combination is fused, the pulse sequence used to achieve this successfully will be stored in the LUT and the memory 21, together with information regarding the complete set of fusion parameters at which the desired response was observed. The processor 20 can then use the indication of the response to select a pulse sequence from the LUT.

Alternatively, the pre-programmed pulse sequences could be stored in the LUT in accordance with each particular type of target and partner cell combination. Again, this information will need to be determined initially. However, by storing the pulse sequence each time a new target and partner cell combination is fused, this allows the processor 20 to select a pulse sequence at step 310 in accordance with cell types provided by the user at step 300.

In any event, the electric pulse sequences applied to the cells to cause the cells to fuse by DEP at step 320.

At step 330, whilst the cells are still in the fusion well 44 the user examines the both morphology and the electrical behaviour of the cells to determine if they have fused to create a fusate cell. If the morphology and behaviour appear favourable to fusion then the fusate is transferred using the pipette 33 to the recovery well 45 at step 360. The initial stages of cell fusion only take a few minutes, typically under ten for most type of cells and accordingly, the user can simply view the cells on the I/O device 22 and determine from this whether the fusion process has been successful. If it is determined that the cells have not fused at step 340, the user simply discards the unfused cells with the pipette 33 at step 350, and returns to select new cells at step 210.

Once placed in the recovery well 45 the fusate cell is left for approximately 45 minutes before again being checked at step 370. During this time, the cell is held in suspension in a suitable culture medium to encourage cell growth. If it is determined that the fusate cell has not completely fused at step 380 then the user discards the unfused cells using the pipette 33 at step 390, and selects new cells at step 210.

Otherwise, the user transfers the fusate cell to a respective hybrid well 46 using the pipette 33 at step 400. The fusate cell is incubated in the hybrid well at step 410, with the cell being monitored after and during the incubation process at step 420, to determine if the fusion has been successful.

Pulse Sequences

As described briefly above, different pulse sequences may be used to control the fusion of the two cells. The generation of different pulse sequences is achieved by having the processor 20 control the signal generator 13 in accordance with pre-determined pulse sequences stored in the memory 21. The pulse sequences are generally stored in data arrays and associated parameters in an LUT, as outlined above or calculated using suitable equations and data arrays at the point of fusion. The processor 20 extracts the necessary parameters and the like stored in the memory 21 and transfers this information to the signal interface 25.

In this example, the signal interface 25 is in the form of an arbitrary signal generator or the like, which uses the determined parameters to define a desired pulse sequence. The signal generator therefore generates a signal representative of the pulse sequence and transfers this to a high frequency signal amplifier, allowing the desired pulse sequence to be transferred to the electrodes 35 as required.

It will be appreciated that other forms of pulse sequence generation can also be used.

In any event, an example of different electrical pulse sequences that may be used for fusing cells will now be described. In each of these examples, the functions are defined in the temporal domain, t

The basic pulse sequence profiles may be defined in terms of the equations:
y₁(t)=A sin(ωt)<t₁
y₂(t)=C(t)t₁<t<t₂
y₃(t)=B sin(ωt)>t₂
where: A is a constant
   B is a constant (and may be equal to A)
   C(t) is the function describing the pulse.

C(t) is typically based on one of the following functions, although it will be appreciated that this is not essential:
C₁(t)=±K
C₂(t)=Q exp(−αt)
C₃(t)=Q exp(αt)
C₄(t)=Q sin(ξt)
where: K Q, α and ξ are constants.

Basic pulse sequences can be combined and overlaid to create complex sequences, some examples of which are listed below and are shown in FIGS. 7A to 7G.

FIG. 7A shows a first example of a Basic DC Fusion Pulse Sequence consisting of 2 unipolar square pulses, separated by sinusoidal waves. The equations used to govern the generation of these pulse sequences are as follows:
y₁(t)=A sin(ωt)<t₁
y₂(t)=+K t₁<t<t₂
y₃(t)=B sin(ωt)t₂<t <t₃
y₄(t)=+K t₃<t<t₄
y₅(t)=A sin(ωt)>t₄

FIG. 7B shows a second example of a Basic DC Fusion Pulse Sequence consisting of a bipolar square pulse, separated by sinusoidal waves. The equations used to govern the generation of these pulse sequences are as follows:
y₁(t)=A sin(ωt)<t₁
y₂(t)=+K t₁<t<t₂
y₃(t)=−K t₂<t<t₃
y₄(t)=A sin(ωt)>t₃

FIG. 7C shows a third example of a Basic AC Fusion pulse consisting of a sinusoidal (of differing frequency) of increased amplitude and differing frequency separated by sinusoidal waves. The equations used to govern the generation of these pulse sequences are as follows:
y₁(t)=A sin(ωt)<t₁
y₂(t)=Q sin(ξt)t₁<t<t₂
y₃(t)=B sin(ωt)t₂<t<t₃
y₄(t)=Q sin(ξt)t₃<t<t₄
y₅(t)=A sin(ωt)>t₄

FIG. 7D shows a fourth example of a Basic DC and exponential pulse separated by sinusoidal waves. The equations used to govern the generation of these pulse sequences are as follows:
y₁(t)=A sin(ωt)<t₁
y₂(t)=+K t₁<t<t₂
y₃(t)=K+Q exp(−αt)t₂<t<t₃
y₄(t)=A sin(ωt)>t₃

FIG. 7E shows a fifth example of a Basic DC and exponential pulse separated by sinusoidal waves. The equations used to govern the generation of these pulse sequences are as follows:
y₁(t)=A sin(ωt)<t₁
y₂(t)=+K t₁<t<t₂
y₃(t)=K−Q exp(αt)t₂<t<t₃
y₄(t)=A sin(ωt)>t₃

FIG. 7F shows a sixth example of a Basic DC pulse sequence convoluted with a linear curve. The equations used to govern the generation of these pulse sequences are as follows:
y₁(t)=A sin(ωt)<t₁
y₂(t)=+K\textcircled{x}(−βt)t₁<t<t₂
y₃(t)=B sin(ωt)t₂<t<t₃
y₄(t)=+K\textcircled{x}(−βt)t₃<t<t₄
y₅(t)=A sin(ωt)>t₄

Note. An extra DC pulse is shown in FIG. 7F for clarity.

FIG. 7G shows a seventh example of a Basic DC pulse convoluted with an exponential decay curve. The equations used to govern the generation of these pulse sequences are as follows:
y₁(t)=A sin(ωt)<t₁
y₂(t)=+K\textcircled{x}(Q exp(−αt)t₁<t<t₂
y₃(t)=B sin(ωt)t₂<t<t₃
y₄(t)=+K\textcircled{x}(Q exp(−αt))t₃<t<t₄
y₅(t)=A sin(ωt)>t₄

Note. An extra DC pulse is shown in FIG. 7G for clarity.

### SPECIFIC EXAMPLE

An outline of the production of a human-human hybridoma using the apparatus of FIG. 1 will now be described. In general the explanation will focus on the following staged of the process.
Preparation of the cells for fusion
Setup of the apparatus for fusion
Manipulation of the cells in readiness for fusion
Electrofusion of the selected pair of cells to obtain hybrid fusates.
   Preparation of the Cells for Fusion.

Peripheral Blood Mononuclear Cells (PBMC) were prepared according to the following protocol: Buffy Coats are obtained from healthy donors (sero-negative for HIV, Hep-B, Hep-C, HTLV-I and Syphilis) from the Australian Red Cross Blood Bank, Sydney, NSW. PBMC are isolated by density centrifugation on Ficoll-Paque™ Plus (Amersham Pharmacia, 17-1440-03). The B cells are then isolated for fusion. Untouched B cells are isolated from PBMC with MACS B Cell Isolation Kit (Miltenyi BioTec, 469-01) by magnetic depletion of T cells, NK cells, myeloid cells, basophils, platelets and early erythroid cells. A human myleloma cell line, designated F4 was used as the immortal partner cell.

Set-Up of the Apparatus for Fusion

A 20 ml syringe (#1) was loaded with RPMI media (#2) warmed in an incubator (30 mins at 37 C). Using the syringe a large droplet of the warmed RPMI solution was deposited into the centre of a Petrie dish (#3). This dish was then placed on the inverted microscope (Nikon TE2000) such that it was situated beneath the pipette. The pipette having first been sterilized with repeated washings of 70% alcohol/water solution. The pipette was then lowered so that the tip was immersed in the droplet of RPMI. One end of a length of silicon tubing(#4) (with suitable connectors(#5)) was attached to a second syringe and the other end to the pipette. RPMI was then gently drawn into the pipette and through the tubing using the syringe. Care was taken to ensure that no air bubbles formed anywhere along the tubing or in the pipette. Using the RPMI filled syringe, fluid was injected into the nozzle of the piezo electric actuator until it was completely filled and a positive meniscus formed over the nozzle. The second syringe was then gently uncoupled from the silicon tubing. Using the first syringe filled with RPMI the uncoupled end of the silicon tubing was topped with fluid until a positive meniscus over the mouth of the connector. The tubing was then coupled to the piezo electric nozzle 54.

Each pipette nozzle 54 is drawn from capillary tubing (120 μm inner diameter) from (#7)

The electrodes 35 were then aligned using a graticule until they were spaced ˜400-500 μm apart.

The previously prepared partner cells were then transferred to a single well in a 96 well plate (#6) and the lymphocytes were deposited in a separate well in the same plate.

The pipette was then inserted into the well containing the partner cells and a suitable partner cell selected. This (single) cell was then transferred to a fresh well containing RPMI+10% fetal calf serum; FCS. The pipette was then inserted into a well containing the previously sorted B lymphocytes specific to the target antigen. A suitable B lymphocyte for fusion was then selected. Returning to the previous well the lymphocyte cell was expelled from the pipette beside the partner cell. Both cells were then visually inspected for their suitability for fusion.

Manipulation of the Cells Prior to Fusion

The pipette was then used to transfer both cells into a well containing an enzyme solution of 1% pronase plus a sorbitol solution of appropriate pH and osmolarity. The cells were immersed in this medium for five minutes before being ‘washed’ in the fusion medium (which is generally formed from a sorbitol solution of appropriate pH and osmolarity) by gently inhaling and expelling them through the pipette aperture 58 in order to allow them to acclimatise to the changed environment. Once the cells had adjusted to the change in osmolarity the pipette was then used to hydrodynamically arrange the cells so that they were within 5-10 μm of each other. The pipette was then removed from the well.

The electrodes 35 were inserted into the well and arranged so that the previously arranged cells lay centred and co-linearly between them. Each electrode is constructed from a nickel alloy wire of 180 μm diameter manufactured by the Californian Fine Wire Company, California, USA The configuration of the electrodes, their shape and their proximity to the cells are specifically designed so that a suitable electric field pattern can be generated in order to induce DEP between the cells.

The electrodes were connected through an amplifier to the arbitrary signal generator and a series of voltages conforming to different waveforms, previously defined by the user, were applied. The first waveform applied to the electrodes was sinusoidal and had a frequency of 500 kilohertz and an amplitude, post amplifier, of approximately 6V peak to peak. Through phenomena known as dielectropherisis, whereby neutral particles become polarised in the presence of an alternating, non-uniform, electric field, the cells experienced a force of attraction that caused them to coalesce.

The amplitude of the field was then increased to 15V peak to peak for a period of 5 seconds ensure that good membrane contact was made between the cells. In this increased field there was a slight drift of the cells towards the upper electrodes, and to counter this the stage of the microscope was adjusted relative to the electrodes to correct and retain the cells position between the electrodes.

Electrofusion of the Selected Pair of Cells to Obtain Hybrid Fusates.

Once the cells were suitably arranged a field pattern conforming to the fusion pulse sequence was applied. In this instance the fusion pulse sequence consisted of two pulse trains, each train consisting of 2 DC pulses, of amplitude 90V, (resulting in an electric field of approximately 180 kV) each being of 80 μs duration. The pulses were separated by a duration of 100 μs, and each train was separated by 500 milliseconds, during which in the intervening time a DEP field was applied in order to keep the cells in good contact. Post fusion pulse sequence, an increased DEP field was applied in order to maintain good contact between the cells whilst the cells fused.

Recovery of the Cells to Growth Medium

The electrodes 35 were the retracted from the fusion well, and the pipette 33 was inserted and manipulated so that the newly created fused cells were in the vicinity of the pipette aperture 58. The cells were then inhaled into the pipette and the pipette retracted from the well. In this fashion the cells were transferred to a fresh well containing hybridoma growth media (RPMI+10% FCS). The newly fused cells were the only cells that were present in this media.

Automation

The above description focuses on manual use of the apparatus, in which positioning of the cells, electrodes and pipette are controlled in accordance with commands input by the user.

However, alternatively the processing system 10 can be adapted to control the apparatus automatically. In order to achieve this, the processor 20 executes image recognition applications software stored in the memory 21. This allows the processing system to use images received from the imaging system 11 to determine the position of cells within the wells 41, . . . 48, as well as to determine the position of the electrodes 33 and the pipette 33.

From this, it will be appreciated that the processor 20 and be programmed to perform the procedure outlined above automatically. Accordingly, the processing system will be adapted to automatically select target and partner cells in accordance with the appearance of the cell in the image. The cells will then be placed in the fusion well 44 to allow the fusion to be performed. Again, during this process the processor 20 will control the position of the cells and the electrodes.

The processor then determines the pulse sequence to be applied to the cells, and applies the pulse sequence via the electrodes 35. Once this is completed the processor 20 can monitor the cells to determine if the fusion process is successful.

It will be appreciated that this may advantageously be achieved using the apparatus described in our copending application entitled “Cell Recovery”.

Modifications

Experiments have indicated that practically as few as one in seventy fused cells retain the genes needed for mitosis and of these stable cell lines a much smaller fraction go onto secrete a protein of interest. It is therefore desirable to have an apparatus that combined the benefits of single cell fusion along with high with a throughput of fused cells. Examples of apparatus providing techniques for improving the throughput of the above described apparatus will now be described.

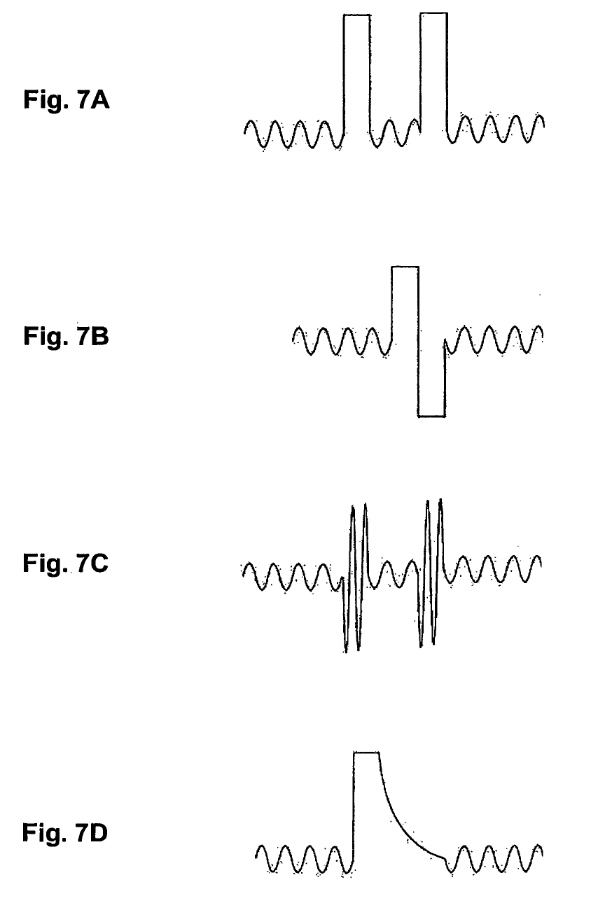

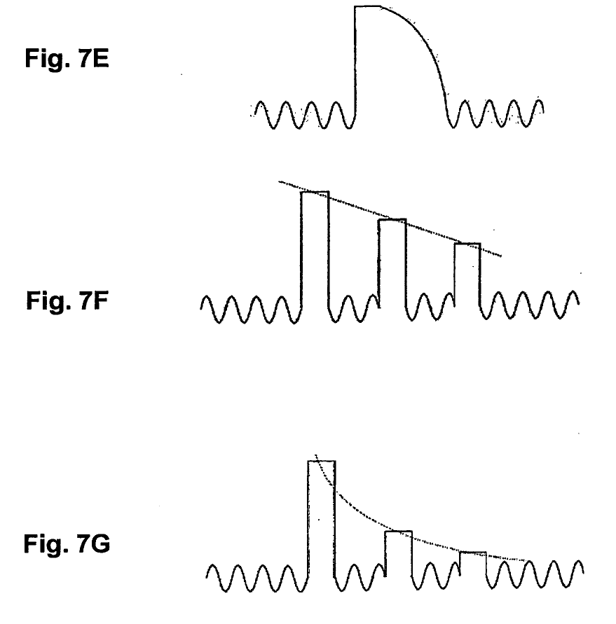

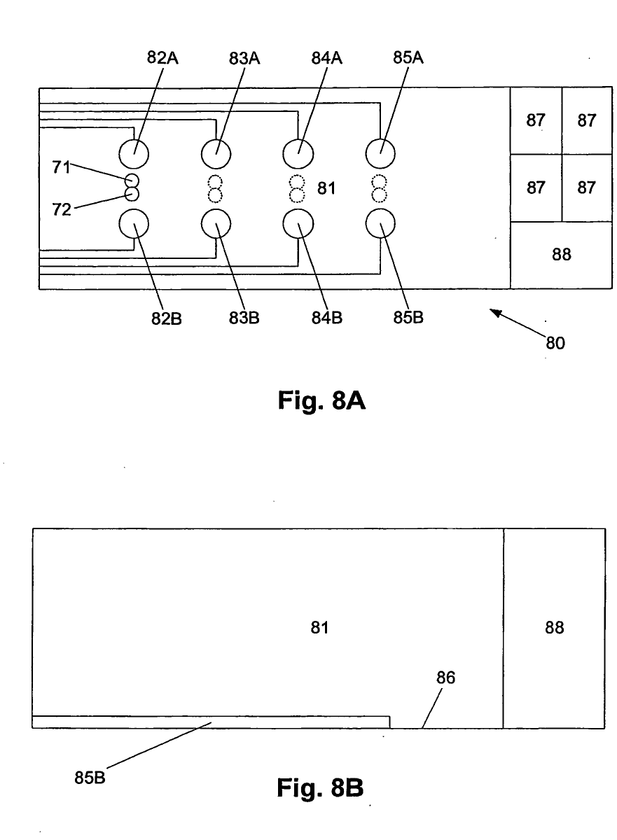

A second example of apparatus suitable for fusing cells will now be described with reference to FIGS. 8A and 8B.

In particular, the apparatus is substantially the same as the apparatus described above with respect to FIGS. 1 to 3. However, in this example, the apparatus includes a modified well array 40 having electrodes incorporated therein. Accordingly, the electrodes 35 are not required with the electrode system 15 utilising the electrodes within the well array as will be described in more detail below.

An example of the modified well array is shown in FIG. 8A. As shown, the well array 80 includes a fusion well 81. Mounted within the fusion well 81 are a number of pairs of electrodes 82A, 82B, 83A, 83B, 84A, 84B, 85, 85B. Although only four pairs of electrodes have been shown in this example, it will be appreciated that a greater number of electrodes may be used if an appropriately sized fusion well is provided.

The electrodes are typically formed from gold plated to a thickness of ˜2 ˜m onto a lower surface 86, as shown in FIG. 8B. The well array may also provided with one or more recovery wells 87, 88 as shown.

In use, the predetermined pulse sequences may be applied to the cells 71, 72 to be fused using the electrode pairs to 82, 83, 84, 85 as shown.

In use, the user will select the cells 71, 72 to be fused and position the cells between a respective pair of electrodes 82 using the pipette, as described above. Once the cells 71, 72 are positioned between the electrodes 82, the predetermined pulse sequence may be applied to the electrodes to thereby cause the cells to fuse in the manner described above.

From this it will be appreciated that four pairs of cells may be positioned in the fusion well 81 at any one time, as shown by the dotted lines. Whilst it is possible to fuse the four pairs of cells simultaneously, it is possible for the field sequence generated each pair of electrodes 82, 83, 84, 85 to interfere. Accordingly, in some cases it is preferable for each pair of cells to be fused in sequence.

In order to achieve this, the processing system 10 can be adapted to apply a first predetermined pulse sequence to the electrodes 82, followed by a second predetermined pulse sequence to the electrodes 83, etc. It will therefore be appreciated that different field sequences may be applied to different pairs of electrodes to allow different cells to be fused within the same recovery well.

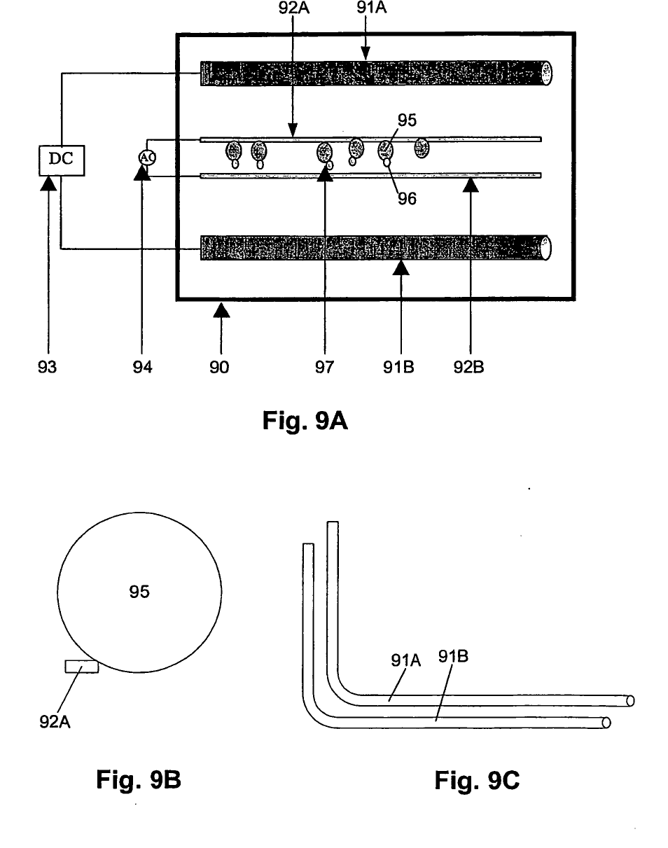

A third example of apparatus for fusing cells will now be described with reference to FIGS. 9A, 9B and 9C. In particular, FIG. 9A shows a fusion well 90 having a first pair of electrodes 91A, 91B and a second pair of electrodes 92A, 92B. In use the electrodes 91 are coupled to a first signal generator 93 with the electrodes 92A, 92B being coupled to a second 94. In this case the first and second signal generators replace the single signal generator 13 shown in FIG. 1, so that the signal generators 93, 94 are coupled to the processing system 10, via an appropriate interface 25, to allow their operation to be controlled.

In this example, the electrodes 92A, 92B are used to generate a DEP field which is adapted to induce a dipole in cells provided at an appropriate location within the fusion well 90. This is used to attract the cells to a selected one of the electrodes 92A, 92B, thereby allowing the cells to be positioned accurately within the fusion well.

Accordingly, in use, the AC signal generator 94 will be activated to generate a DEP field. A pipette is then used to insert cells 95 into the fusion well 90, in a manner similar to that described above. In this case, the cell 95 are attracted to the electrode 92A, and will therefore align as shown. It will be appreciated that this inherent attraction reduces the accuracy with which cells must be positioned within the fusion well 90, compared to in the techniques outlined above, and will operate to retain the cells 95 in position during subsequent processing.

As shown in FIG. 9B although the cell may contact the electrode 92A, as the electrode is typically formed from a layer of gold plated onto the bottom of the fusion well 90, the point of contact between the cell 95 and the electrode 92A will typically only be very small. Thus, since these electrodes are only of the order of a micrometer high, and are only used to supply the relatively low power DEP field and not the higher power fusion pulse, as will be described below, the cells will not be damaged by the procedure, and will be easy to recover from the fusion well 90.

In any event, with a number of first cells 95 positioned in the chamber 90 a number of second cells 96 may be positioned adjacent the first cells 95. In use the dipole induced in the first cells 95 will attract the second cells 96 to form a number of cell pairs, as shown in FIG. 9A.

Once the required cells are held in position within the fusion well 90, a fusion pulse can be applied to the electrodes 91A, 91B via the first signal generator 93. This fusion pulse may consist of a simple DC current applied to the electrodes 91A, 91B, or may be formed from a more complex waveform. Similarly, the electrodes 92A, 92B are used to generate a DEP field in accordance with signals from the second signal generator.

Thus, as shown in the signals shown in FIGS. 7A-7G the overall electric field experienced by the cells consist of a generally alternating DEP field, with a superimposed fusion pulse formed from a substantially DC field. In this example, instead of this being achieved using a single set of electrodes, the fusion pulse is produced by the first set of electrodes 91 with the DEP field being produced by the second set of electrodes 92.

In this example described, the electrodes 91 can be provided in the cell as fixed electrodes. Alternatively however the electrodes may be positioned in the cell once the cells 95, 96 are in place. This has a number of advantages and in particular will avoid stray currents in the electrodes disturbing the cell placement. An example of the electrodes used in such an arrangement are shown in FIG. 9C.

This arrangement has a number of benefits.

Firstly, allowing the first cells 95 to be placed in a DEP field generated by the electrodes 92 allows the cells to be arranged far more easily in the fusion well 90. In particular, as mentioned above, the cells 95 are held in place by the DEP field, thereby ensuring that they do not move after placement when further cells are added. This allows the cells to be placed as close as five cell diameters apart (although this is not shown in the figure for clarity) allowing a large number of cells to be aligned accurately in the fusion well 90.

Secondly, the second cells 96 are attracted to the first cells 95 by the generated DEP field, thereby causing the cells to naturally align to form cell pairs, as shown at 97. This vastly aids the practical speed with which cell pairs can be formed at correct locations within the fusion chamber 90. In particular, this allows a number of cell pairs to be formed in a relatively short space of time such as a couple of minutes, even using manual operation of the pipette.

Thirdly, as the fusion pulse is provided by the first electrodes 91, the cells will not be damaged by contact with the second electrodes, thereby allowing the cells 96, 96 to be inserted into the fusion well 90 without requiring that they are positioned near to, but out of contact with the electrodes. As the cells are retained in position well away from the first electrodes 91, this allows a higher field strength to be used for the fusion pulse, which in turn increases the chances of successful cell fusion.

To further enhance this, the DEP field generated by the electrodes 92 can be momentarily increased (˜50 ms) as the fusion pulse is generated between the electrodes 91. The purpose of this is to increase the strength of the dipoles generated in the cells 95, 96, thereby urging the cells together with an increased force, to ensure good membrane to membrane contact between the cells during fusion. This helps increase the chances of successful cell fusion. Once the fusion pulse is applied the increased DEP field can be maintained for a short time after pulsing in order to further aid fusion.

Finally, a further beneficial result of this configuration is that a number of cell pairs 97 can be arranged in the fusion well 90 and exposed to substantially identical field conditions. This allows a batch of cells to be prepared having substantial identical fusate properties. This helps ensure consistency of the fusate, and allows batches of fused cells to be produced for experimental purposes.

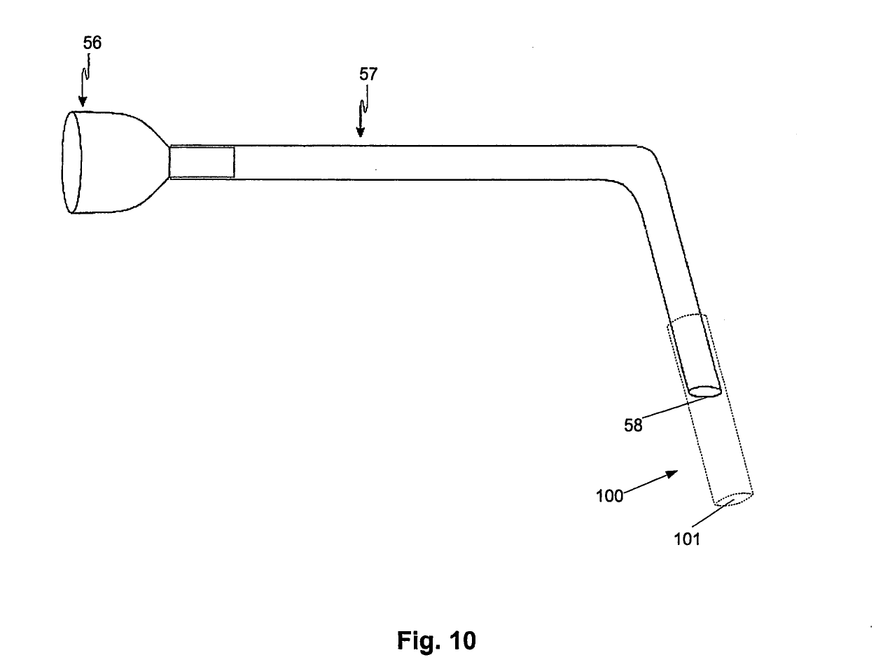

A fourth example of apparatus for fusing cells will now be described with reference to FIGS. 10 to 12.

In this example, apparatus similar to that in FIGS. 1 to 3 is again used with one of the electrodes 35 being replaced by an electrode provided on the pipette 33. An example of the pipette is shown in FIG. 10.

As shown, the pipette is modified by the inclusion of an electrode 100 formed from a cylindrical tube 101, and which is coupled to the nozzle 57. The electrode 100 is coupled to the nozzle 57 such that the aperture 58 is contained in the tube 101 as shown.

In use, the pipette may be used substantially as described above to draw in an expel fluid through the port. This can be used to recover cells from a well allowing the cells to be placed in a fusion well, as described above.

In this example, the fusion well will additionally contain a second electrode. The second electrode may be a separate electrode similar to one of the electrodes 35 shown in FIG. 2. The cells can then be positioned between the electrode 100 and the electrode 35. The signal generator is used to apply a predetermined pulse sequence to the electrodes 100, 35, allowing the cells to be fused as described above.

Alternatively, the electrode may be provided on the underside of the fusion well, in a manner similar to that shown in FIGS. 8A and 8B.

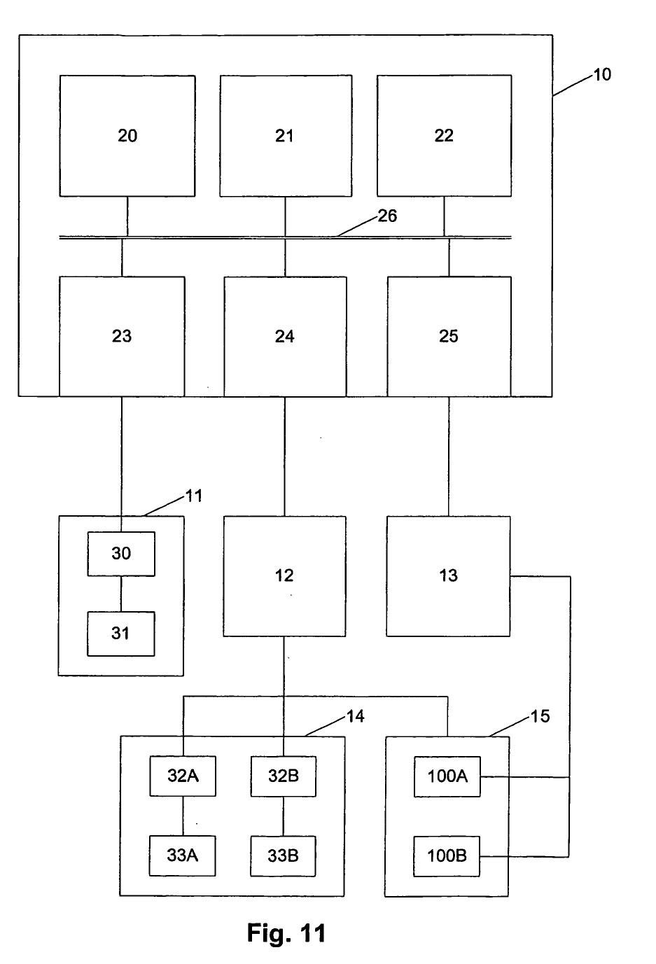

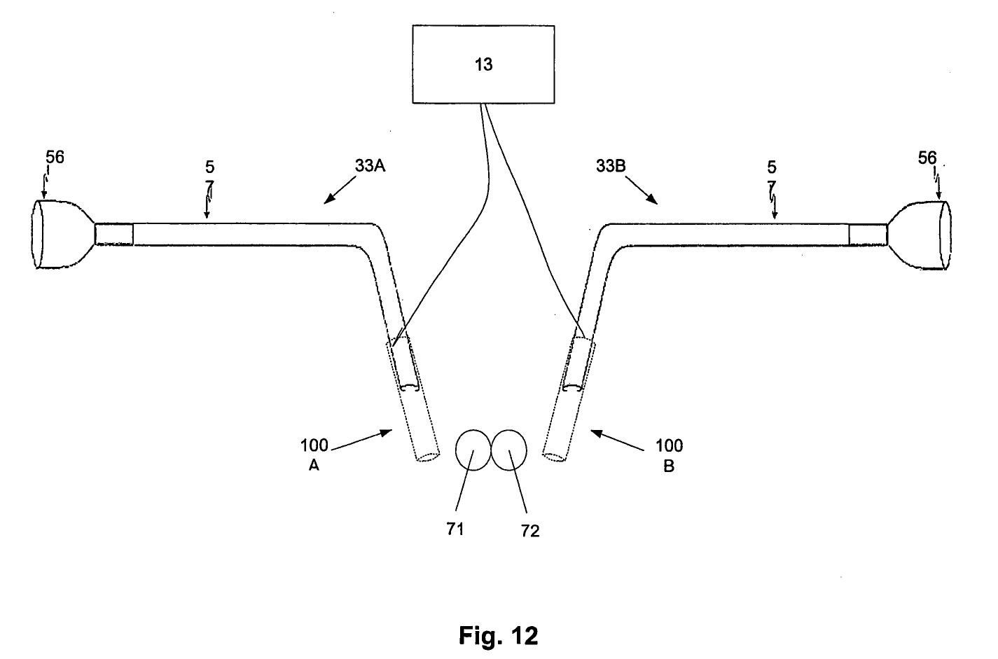

As a further option, a second pipette 33B may be provided with a respective electrode 100B. The resulting apparatus configuration is as shown in FIGS. 11 and 12, with the pipette system 14 being formed from two drive systems 32A, 32B and two pipettes 33A, 33B, as shown Accordingly, in this example, electrodes 100A, 100B provided on the pipettes 33A, 33B, form the electrode system 15.

In any event, an electric field can be generated between the two electrodes 100A, 100B to allow cells 71, 72 to be fused in the manner described above.

It will be appreciated that the provision of a second pipette provides a number of additional advantages.

In particular, each pipette 33A, 33B is used to position respective cells 71, 72 adjacent each other by positioning the first cell 71 using the first pipette 33A, and then positioning the second cell 72 using the second pipette 33B. Once the cells are appropriately positioned, a pulse sequence can be generated between the two electrodes 90A, 90B, thereby causing the cells to fuse.

A number of additional developments can also be implemented for the pipettes. These include the provision of radiation sources such as lasers, LEDs, or the like, and appropriate detectors.

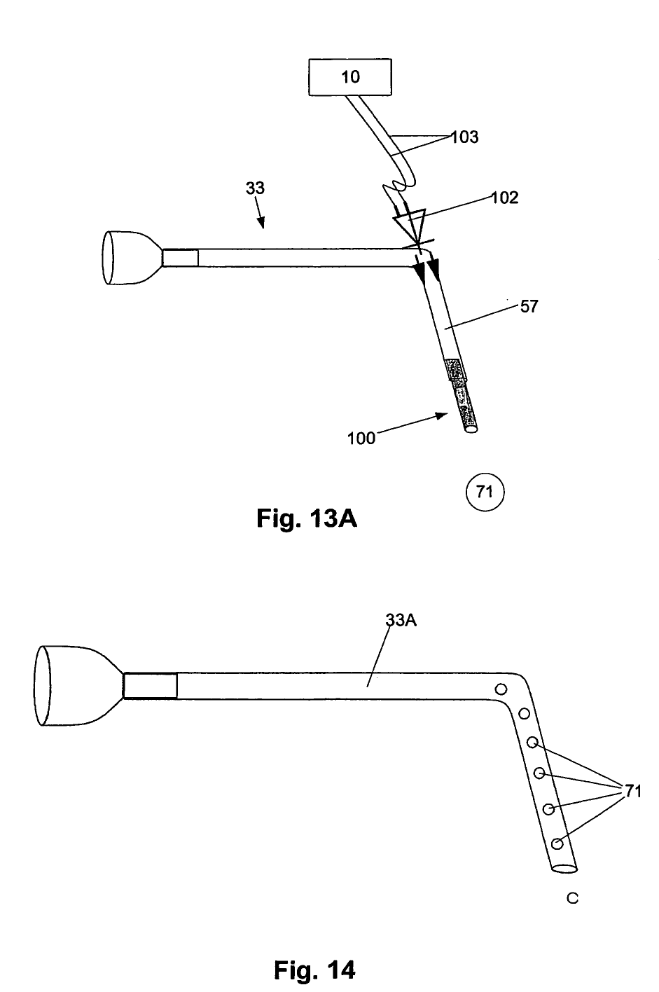

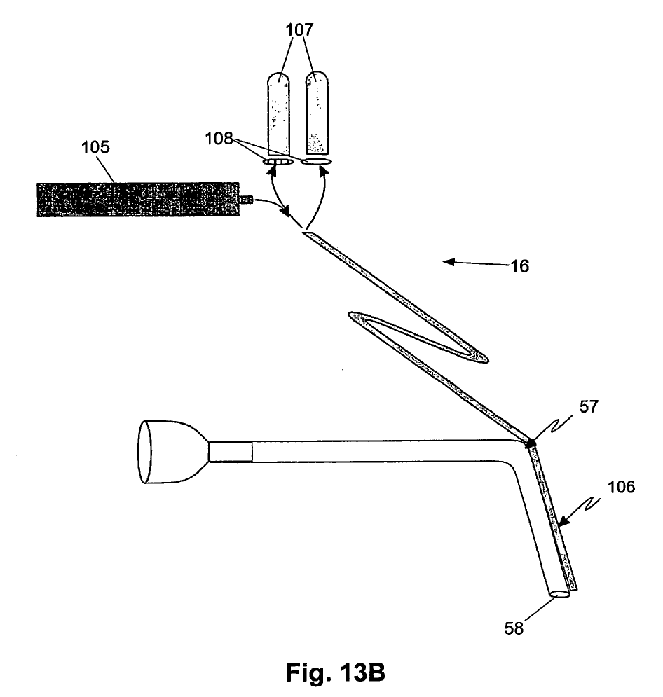

An example of this is shown in FIG. 13A. As shown, the pipette 33 includes an LED 102, arranged to direct radiation along the nozzle 57 and through the aperture 58, and electrode 100, as shown. The LED is typically coupled to the processing system 10, via leads 103, to allow the processing system to selectively activate the LED as required. This allows a cell 71 adjacent the aperture to be exposed to radiation.

This can be performed for a number of reasons. Thus, for example, this may be performed to provide simple illumination of the cells. In particular, illuminating the cells provides a increased contrast between the cell and surrounding fluid medium, thereby making it easier for the camera to resolve the cells. This in turn makes images of the cells presented to the user easier to see, as well as making automated detection of the cells easier.

In addition to this, the illumination allows cells to be labelled with fluorescent markers or the like, to allow the detectors to detect the cells having predetermined properties as described for example in our co-pending Patent Application entitled “Cell Recovery”. In this case, visible radiation from an LED may not have sufficient power to cause the markers to fluoresce. This may be overcome achieved through the use of an LED operating at ultra-violet wavelengths. Alternatively, this may be achieved using a laser based system as shown in FIG. 13B.

In this example, a laser 105, or other radiation source such as a UV burner with suitable filters, is coupled to an optical fibre 106. The optical fibre 106 is coupled to the pipette nozzle 57, using appropriate fixing means, such as a rubber tube (not shown). The optical fibre 106 is also coupled to detectors 107, such as photo-diode tubes, via suitable filters 108.

In use, radiation emitted from the laser is used to expose cells. Any radiation subsequently reflected from, or emitted by the cells, which impinges on the fibre optic cable 106 is transferred to the detectors 107. The processing system analyses signals from the detectors and uses these to select and remove individual cells from a group of cells held in suspension.

In addition to this, in the example of the system shown in FIG. 16, each pipette 33A, 33B could be provided with an LED 102A, 102B having a different wavelength. This allows the cells to be exposed by different wavelengths of radiation either to allow cells having different properties to be detected, for example through the use of alternative markers, or to allow the processing system 10 or the user to determine which pipette the respective cell is near.

This also allows the processing system to use the imaging system 11 to determine from the wavelength of the radiation exposing each cell 71, 72, which pipette 33A, 33B is adjacent the cell. This also allows cells 71, 72 having different predetermined properties to be detected, by arranging for each cell to respond to a respective wavelength of radiation, for example by the use of appropriate labels.

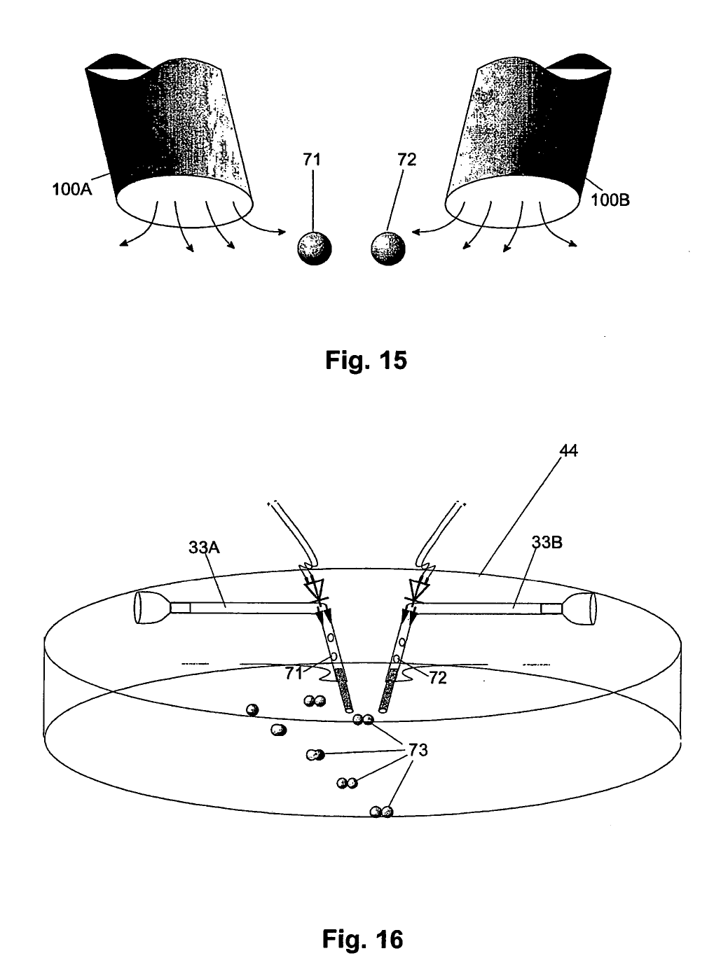

This aids in automating the system and provides for a method that allows a number of cell pairs to be rapidly fused as follows:
1. Multiple cells in a source well are exposed to radiation from the LED 102A;
2. Cells 71 having predetermined properties are detected by the processing system 10 and drawn into and stored in the pipette 33A, as shown in FIG. 14.
3. Multiple cells in a source well are exposed to radiation from the LED 102B;
4. Cells 72 having predetermined properties are detected by the processing system 10 and drawn into and stored in the pipette 33B, in a similar fashion.
5. Both pipettes 33A, 33B are inserted into a fusion well 44.
6. A respective one of each cell type 71, 72, is expelled from each pipette 33A, 33B at the same time, such that hydrodynamic forces draw the cells 71, 72 together as shown in FIG. 15.
7. The processing system detects the positions of the cells using the imaging system 11 such that when the cells are expelled from the respective pipette 33A, 33B the fluid flow is truncated.
8. A DEP field is applied to draw the cells together between the electrodes, as shown for example in FIG. 12. At this point the cells are pushed together using an increased (amplitude) DEP field to aid membrane contact.
9. The signal generator 13 applies a predetermined pulse sequence to the cells 71, 72 via the electrodes 100A, 100B.
10. The cells are again pushed together using an increased (amplitude) DEP field to aid in fusion.
11. The pipettes 33A, 33B move to a new position within well.
12. Steps 6-11 are repeated as many times as necessary, until a number of fusates 73 are provided as shown in FIG. 16.
13. When all cell pairs have been expelled/fused on of the pipettes travels back through the well recovering the fusates.
14. Fusates are recovered to recovery wells either as single clones or groups.

It will be appreciated that this technique can be implemented without the presence of the electrodes 100A, 100B, for example by suitable modification of the pipette shown in FIG. 3.

There also exist techniques for labelling cells that allows them to be magnetically sorted. In this example, small metal beads are used as markers to identify cells of interest. This is achieved by ensuring that cells having desired properties can be fused to the beads and thereby extracted from a mixture of cells.

This can be achieved for example by coating the beads with an antibody of interest and then mixing the beads into a culture of cells. Cells that are expressing the appropriate receptor on the surface bind to the beads. The culture is then filtered through a tube, placed in an external magnetic field containing thousands of small beads that attract and hold the labelled cells, whilst allowing the unlabelled cells to be washed through and discarded. Once the external magnetic field is removed the bound cells can then be washed through the tube and isolated as desired.

It will be appreciated that this may be achieved on a smaller scale using a pipette modified to incorporate an electromagnet.

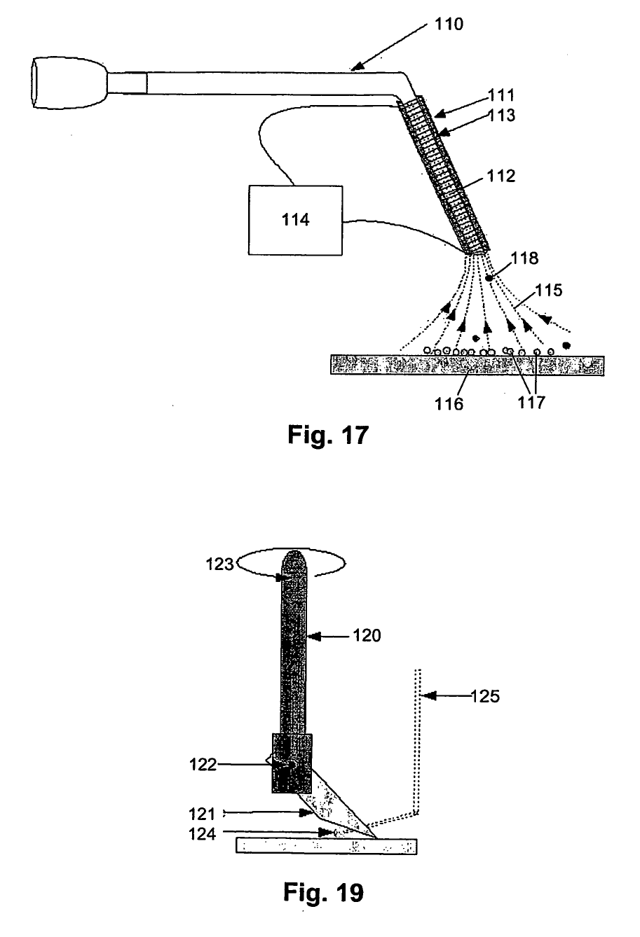

An example of a suitably modified pipette will now be described with reference to FIG. 17. In this example, the pipette shown generally at 110 includes a graphite layer 111 positioned around the pipette nozzle 112. A number of cooper windings 113 are provided around a graphite core to form an electromagnet. In use the copper windings are coupled to a DC signal generator shown generally at 114, so that the windings act as a solenoid to generate a magnetic field represented by the field lines 115.

The copper windings may be provided in a number of layers depending on the implementation, and may be embedded in a layer of epoxy in order to prevent electrolysis from occurring.

The ends of the wire are connected to a variable DC signal generator and a resistor (R). Passing a current through the wire (taking account of Lenz's Law) will induce a magnetic field, the strength of which is proportional to the applied DC Voltage (V), as given by the equation:

$$B = nuI = \frac{nuV}{R}$$

where: n=the number of turns per unit length
   u=the permeability of free space.

In use, the pipette is positioned near a number of cells which may suspended in a fluid medium or resting on a substrate 116 as shown at 117. In this case, at least some of the cells are attached to appropriate magnetic markers, such as the beads outlined above.

In use, the metal particles, and hence the cells they are attached to, will be attracted into the magnetic field and can therefore be drawn into the pipette in the normal way. This allows cells coupled to the magnetic markers, and hence cells having certain properties to be selected.

It will be appreciated that cells with a higher density of receptors (a higher number of magnetic markers), should have a larger force exerted on them than cells with less receptors for the same magnetic field strength Therefore as the DC voltage is increased, a larger number of cells should be drawn into the magnetic fields influence. This field gradient can allow for a further sorting criteria.

In order to ensure no wanted cells have been collected, it is possible to flush out the pipette by expelling fluid from the nozzle. In this case, any cells not bound magnetic markers will be expelled from the pipette together with the fluid, whilst the cells bound to markers will be held in place by the action of the magnetic field. In this case, when the selected cells are to be expelled, the magnetic field can be deactivated, allowing the cells and attached markers to be expelled in the normal way.

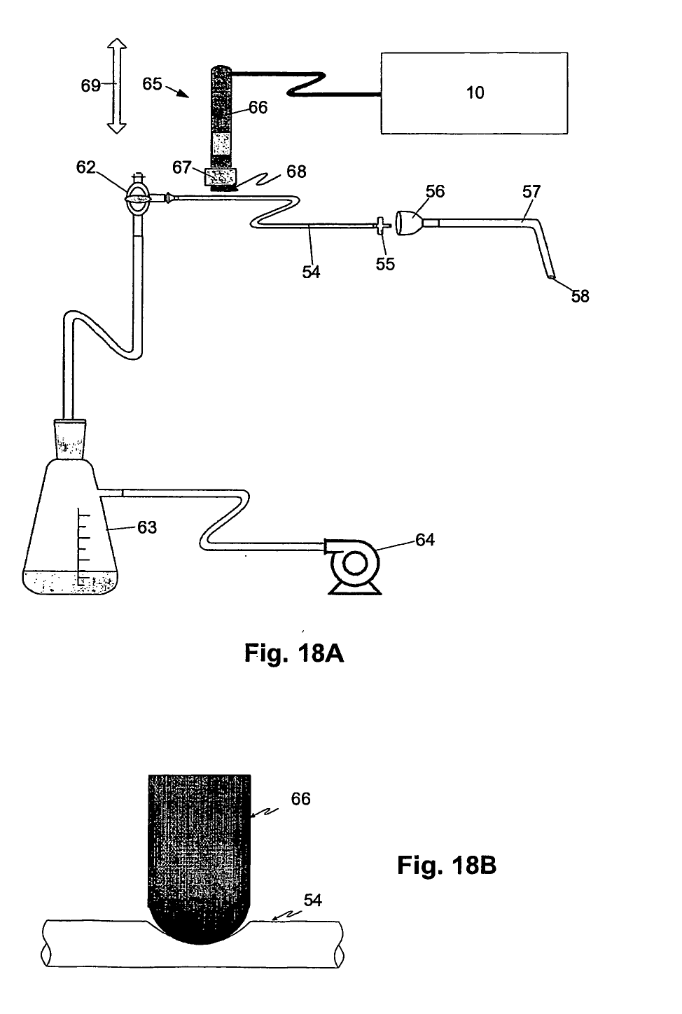

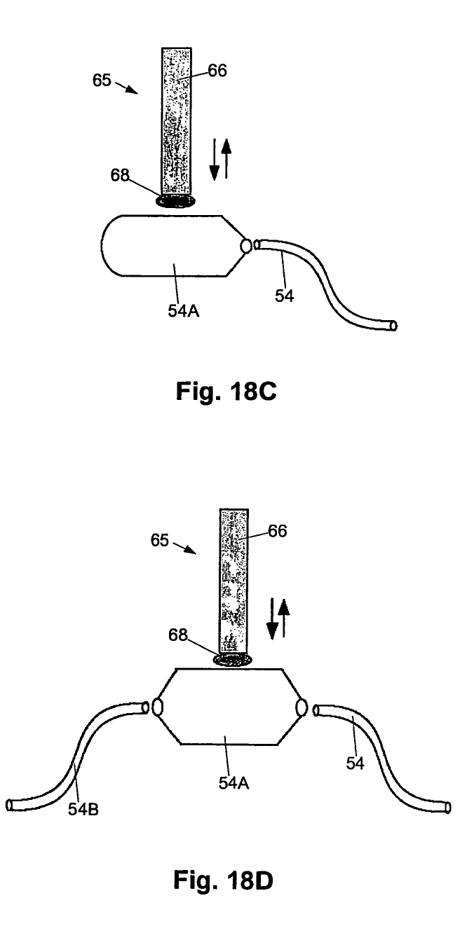

A further development, is for an alternative form of actuator to be used. An example of this form of actuator is shown in FIGS. 18A, 18B.

As shown, in this example, the tube 54 is connected via a stopcock 62 and a reservoir 63 to a pump 64. An actuator 65 is positioned adjacent the flexible tube 54, to allow the tube to be clamped as shown in FIG. 18B.

It will be appreciated from this that any form of actuator, such as a solenoid, may be used. However, in this example, the actuator is formed from a threaded screw drive 66, coupled a DC or stepper motor 67, which forms part of the drive system 32. In use, this allows the actuator to be moved in the direction of the arrow 69, an amount of ±5 mm.

The actuator tip can have a piezo electric stack 68 coupled thereto, to allow fine control (displacement of ±20 μm) of the end of the actuator. Again, the piezo stack forms part of the drive system 32.

In use, the pipette is loaded with a suitable fluid medium by placing the aperture 58 into a container that has sufficient fluid to fill the system. The pump or other such means of drawing fluid through the system is activated and fluid is drawn through the pipette nozzle 57. When the system is loaded and there are no air bubbles present in the tubing, the stopcock 62 is closed to prevent further fluid flow, and the pump 64 turned off.

Whilst the aperture 58 is still immersed in the fluid medium, the actuator 65 is adjusted such that the silicon tubing 54 is compressed to about half its diameter, as shown in FIG. 2B. Thus, in use, with the port 41 positioned in fluid in a well causing the actuator 65 to move in the direction of the arrow 69 compresses or releases the tubing 54 which, in turn, either expels or draws in fluid through the port 41. This allows cells to be recovered from a well as described above with respect to the pipette of FIG. 3.

Variation on this are shown in FIGS. 18C and 18D. In these examples, the actuator 65 is positioned adjacent a bladder 54A provided in the flexible tube 54. In this case, the bladder has a larger cross sectional area than the tube and will therefore contain a greater volume of fluid per unit length compared to the tubing 54. This has two main benefits. In particular, the larger cross sectional area provides for a greater range of movement of the actuator. This coupled with the increased fluid volume in the bladder allows for a greater amount of fluid to be displaced when compared to the action of the actuator on the tube 54.

As a result this provides greater control over the amount of fluid expelled or drawn in through the aperture 58, allowing for greater accuracy in retrieving individual cells using the pipette.

In this instance, it will be appreciated that by providing sufficient liquid in the bladder, it is not necessary to provide the stopcock 62, the reservoir 63 or the pump 64 as shown in FIG. 18C. In particular, the bladder and pipette can be filled, with an amount of fluid being expelled from the bladder before the bladder is positioned so as to cooperate with the actuator, thereby allowing the actuator position to be adjusted to allow fluid to be drawn in or expelled through the aperture 58.

Alternatively, the bladder can be connected to a stopcock 62, reservoir 63 and pump 64, by a tube 54B, as shown in FIG. 18D.

Accordingly, the system described above allows individual cells to be easily fused. As the cells are manipulated using the pipette as shown in FIG. 3, this makes the cell manipulation far easier than in the prior art. This therefore helps increase the speed and ease with which fusion of individual cells can be performed. Furthermore, the electrodes need never touch the cells, thereby helping reduce or prevent cell damage prior to or during the fusion process.

In addition to this, the apparatus as a whole is generally less complicated, thereby helping reduce the cost, as well as easing use of the apparatus to perform cell fusion. As a result, fusion using the system described above can generally be achieved more rapidly and cheaper than in the prior art.

A further development that can be utilised within the examples described above is for a cutting tool to be provided to allow cells to be cut, as well as to allow cells that have adhered to the well surface or electrodes to be released. An example of a suitable cutting tool is shown in FIG. 19. As shown, the cutting tool includes a support post 120 having a blade 121 pivotally mounted thereto by a hinge 122 or other appropriate connection.

In use, the post is coupled to a micro manipulator (not shown), to allow the post to be positioned within the respective well. The post can be rotated as shown by the arrow 123, allowing the blade to be positioned above a cell to be cut. If the cell is a free cell 124, the cell will generally be held in place using a pipette, or other suitable manipulator, as shown at 125.

Once positioned, the post is lowered such that the tip of the blade ‘bites’ into the soft plastic of the bottom of the plastic plate. Further lowering of the post will cause the blade to pivot around the hinge 122 and ‘guillotine’ through object, such as the cell, placed in its path. Motion is stopped when the blade has cut through the object of interest and is completely parallel with bottom of plate.

It will be appreciated that the functionality of the different examples described above may be combined in any one of a number of arrangements. This allows for example cells to be selected automatically in accordance with magnetic or radiation sensitive markers. The cells can then be arranged in a fusion well, and fused, with the fusate being automatically retrieved and positioned in a recovery well.

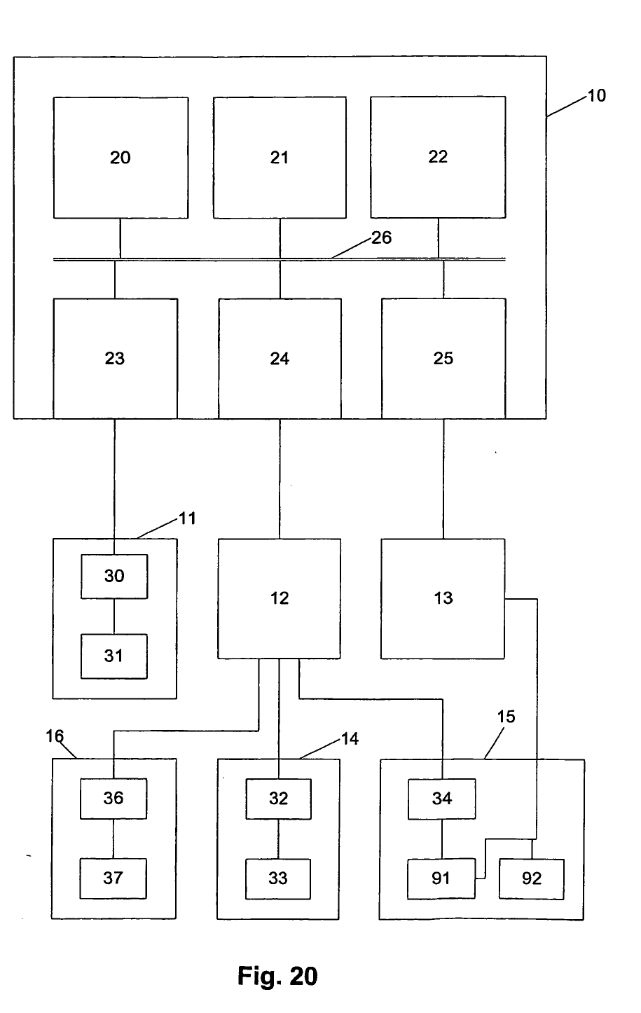

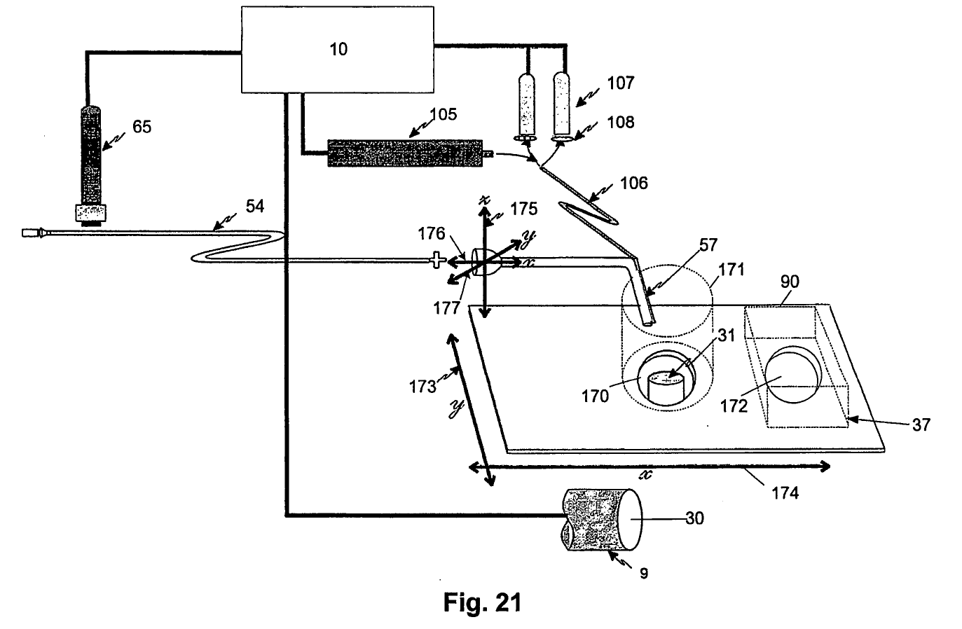

A specific example of apparatus for performing automatic cell selection and fusion will now be described with reference to FIGS. 20 and 21.

As shown in FIG. 20, the control system 12 is further coupled to a stage system 16, including a drive system 36 coupled to a stage 37, with the processing system 10 being coupled to a stimulation system 17. The stimulation system 17 is used to stimulate cells, to allow cells having predetermined properties to be recovered from a group of cells held in suspension in a selection well.

In order to achieve selection the cells are labelled with markers, which are adapted to adhere and or permeate only the cells having the required predetermined properties. The stimulation system 17 stimulates the marker cells and thereby identify the cells having the predetermined properties. It will be appreciated that the stimulation system 17 may be a radiation based system, similar to that described with respect to FIG. 13, or a magnetic based system similar to that described with respect to FIG. 17. The following example will focus on the use of a radiative based approach.

The arrangement of the apparatus is shown in more detail in FIG. 21.

As shown, the stage 37 includes an aperture 170, having the microscope 31 mounted therein. From this it will be appreciated that the microscope 31 is typically an inverted microscope.

In use the stage 37 is adapted to receive a selection well 171 containing the cells to be recovered. The stage will also receive a fusion well 90, positioned over an aperture 172. In use, the selection well 171 is positioned on top of the aperture 170, to allow the camera 30 to obtain an image of the inside of the selection well 171, via the microscope 31. In use, the processing system 10 is adapted to control the drive system 36, to cause the stage 37 to be move in the directions shown by the arrows 173, 174.

This allows a representation of the contents of a selected well can be captured by the processing system 10 using the image interface 23, which is typically a frame grabber or the like. Images may then be used by the processing system to control the drive systems 32, 35 and 36 and the stimulation system 17. Additionally or alternatively, images may be displayed to a user using the I/O device 22.

The pipette is positioned adjacent the stage 37 as shown, to allow the nozzle 57 to be inserted into the well 171. The pipette 33 is coupled to the drive system 32, to allow the pipette to moved with respect to the well, as shown by the arrows 175, 176, 177. Accordingly, the drive system 12 typically includes a micromanipulator system having three independently controlled axis with resolution tolerances and repeatabilities of <5 μm. This system is controlled by dedicated software executed by the processor 20.

In any event, the cells having the predetermined properties are identified by exposing the cells to radiation using the radiation source 105 coupled to the nozzle 57 via the fibre optic cable 106. This allows the detectors 107 to receive radiation emitted by the cells through the fibre optic cable 106 and filters 108, to thereby determine cells having desired properties.

It will be appreciated that in the event that the detection of particles is performed magnetically, this may be achieved as described above with respect to FIG. 17.

The processing system 10 can then control the pipette system. 14 to remove cells from the selection well 171 and place these in the fusion well 90, as described above. During this process a DEP field will be applied to the electrodes 92 to ensure the cells are positioned as required. In addition to this, the stage 37 is moved, to allow the camera 30 to image the fusion well 90 through the aperture 172.

Fusion will then be performed substantially as described above, with the fused cells being removed as required.

Accordingly, the above system describes apparatus suitable for manipulating and fusing cells, and in particular for single cell, mini-bulk or macro-bulk cell fusion. In this regard, the term cells is intended to cover any cells, vectors, particles, molecules, liposomes, and other such vesicles.

In particular, the techniques are particularly advantageous for the purposes of stem cell fusion, as described for example in our copending application “A Method of Cell Therapy” filed on 30 May 2003.

This allows the techniques to be used for generating tissue or cells useful for tissue replacement and/or tissue rejuvenation therapy or a range of organs or tissue areas of the body. The resulting tissue or cells may also secrete or generate a range of cytokines, enzymes, hormones and the like which have improved or more efficacious properties relative to analogous molecules produced from non-fused cells.

In this case, the cells are selected to have desirable properties, such that the generated fusate has properties applicable for a specific purpose.

A suitable list of stem and mature cells and their application for use in transplant and rejuvenation therapy is shown in Table 1. All such stem and mature cells are contemplated and are encompassed by the present invention. As indicated in Table 1, a mature cell may be derived from any human or mammalian or non-mammalian animal or avian species such as from the brain, epidermis, skin, pancreas, kidney, liver, breast lung, muscle, heart, eye, bone, spleen or the immune system. Cells of the immune system include CD4+ T-cells, CD8+ T-cells, NK cells, monocytes, macrophages, dendritic cells and B-bells. It should be noted that the present invention contemplates the fusion of stem cells and mature cells from any source such as a mammal (including human), non-mammalian animal and avian species. Examples of non-human mammals include livestock animals (e.g. sheep, pigs, cows, horses, donkeys, goats), companion animals (e.g. cats, dogs), laboratory test animals (e.g. mice, rats, rabbits, guinea pigs, hamsters) and captured wild animals. A non-mammalian animal includes a reptile, amphibian, insect, arthropod and arachnids. Avian species include poultry, birds (e.g. ducks, emus, ostriches) and aviary birds.

TABLE 1

| Cell type | Application |
|---|---|
| General Stem cell types | |
| Embryonic stem cells | Generation of any tissue for |
| | transplant |
| Somatic stem cells | Generation of tissue for transplant |
| Germ stem cells | Generation of tissue for transplant |
| Human embryonic stem cells | Generation of wide variety of |
| | tissue for transplant |
| Human epidermal stem cells | Generation of tissue for transplant |

Tissue-specific cells:
Includes both somatic stem cells, mature cells and germ line cells

| | |
|---|---|
| Brain | |
| Adult neural stem cells | Generation of neural tissue for |
| | transplant |
| Human neurons | Generation of neural tissue for |
| | transplant |
| Human astrocytes | Generation of neural tissue for |
| | transplant |
| Epidermis | |
| Human keratinocyte stem | Generation of epidermal type |
| cells | tissues such as hair follicles, |
| | sebaceous glands and skin for |
| | transplant |
| Human keratinocyte | Generation of epidermal type |
| transient amplifying cells | tissues such as hair follicles, |
| | sebaceous glands and skin for |
| | transplant |
| Human melanocyte stem cells | Generation of epidermal type |
| | tissues for transplant |
| Human melanocytes | Generation of epidermal type |
| | tissues for transplant |
| Skin | |
| Human foreskin fibroblasts | Generation of skin for transplant |
| Pancreas | |
| Human duct cells | Generation of insulin-producing |
| | cells for transplant |
| Human pancreatic islets | Generation of insulin-producing |
| | cells for transplant |
| Human pancreatic β-cells | Generation of insulin-producing |
| | cells for transplant |
| Kidney | |
| Human adult renal stem | Generation of kidney tissue for |
| cells | transplant |
| Human embryonic renal | Generation of kidney tissue for |
| epithelial stem cells | transplant |
| Human kidney epithelial | Generation of kidney tissue for |
| cells | transplant |
| Liver | |
| Human hepatic oval cells | Generation of insulin-producing |
| | cells for transplant |
| Human hepatocytes | Generation of liver tissue for |
| | transplant |
| Human bile duct epithelial | Generation of liver tissue for |
| cells | transplant |
| Human embryonic endodermal | Generation of liver tissue for |
| stem cells | transplant |
| Human adult hepatocyte stem | Generation of liver tissue for |
| cells (controversial as to | transplant |
| existence) | |
| Breast | |
| Human mammary epithelial | Generation of mammary (breast) for |
| stem cells | transplant |
| Lung | |
| Bone marrow-derived stem | Generation of tissue for transplant |
| cells | including muscle, cartilage, bone, |
| | liver, heart, brain, intestine and |
| | lung |
| Human lung fibroblasts | Generation of tissue for transplant |
| | including muscle, cartilage, bone, |
| | liver, heart, brain, intestine and |
| | lung |
| Human bronchial epithelial | Generation of tissue for transplant |
| cells | including muscle, cartilage, bone, |
| | liver, heart, brain, intestine and |
| | lung |
| Human alveolar type II | Generation of tissue for transplant |
| pneumocytes | including muscle, cartilage, bone, |
| | liver, heart, brain, intestine and |
| | lung |
| Muscle | |
| Human skeletal muscle stem | Generation of tissue for transplant |
| cells (satellite cells) | |
| Heart | |
| Human cardiomyocytes | Generation of heart tissue for |
| | transplant |
| Bone marrow mesenchymal | Generation of heart tissue for |
| stem cells | transplant |
| Simple Squamous Epithelial | Generation of heart and vascular |
| cells | tissue, for example rebuilding |
| | aortic arteries after aneurysm |
| | repairs |
| Descending Aortic | Generation of heart and vascular |
| Endothelial cells | tissue, for example rebuilding |
| | aortic arteries after aneurysm |
| | repairs |
| Aortic Arch Endothelial | Generation of heart and vascular |
| cells | tissue, for example rebuilding |
| | aortic arteries after aneurysm |
| | repairs |
| Aortic Smooth Muscle cells | Generation of heart and vascular |
| | tissue, for example rebuilding |
| | aortic arteries after aneurysm |
| | repairs |
| Eye | |
| Limbal stem cells | Regeneration of the entire corneal |
| | epithelium for transplant |
| Corneal epithelial cells | Regeneration of the entire corneal |
| | epithelium for transplant |
| Bone Marrow | |
| (in some cases be substituted | |
| for cord blood and peripheral | |
| blood as a source of some | |
| of the below stem cells) | |
| CD34+ hematopoietic | Generation of a wide variety of |
| stem cells | tissues for transplant, including, |
| | but not limited to, immune tissue |
| Mesenchymal stem cells | Generation of a wide variety of |
| | tissues for transplant, including, |
| | but not limited to, cardiac tissue, |
| | bone, cartilage, muscle, tendon, |
| | endothelial tissue, vascular tissue |
| | and neural tissue |
| Osteoblasts (precursor is | Generation of bone for transplant |
| mesenchymal stem cell) | |
| Peripheral blood | Generation of a wide variety of |
| mononuclear progenitor | tissues for transplant, including |
| cells (hematopoietic stem | but not limited to cardiac tissue, |
| cells) | bone, cartilage, muscle, tendon, |
| | endothelial tissue, vascular tissue |
| | and neural tissue |
| Osteoclasts (precursor is | Generation of bone for transplant |
| above cell type) | |
| Stromal cells | Generation of a wide variety of |
| | tissues for transplant, including |
| | but not limited to cardiac tissue, |
| | bone, cartilage, muscle, tendon, |
| | endothelial tissue, vascular tissue |
| | and neural tissue |
| Spleen | |
| Human splenic precursor | Generation of spleen tissue for |
| stem cells | transplant |
| Human splenocytes | Generation of spleen tissue for |
| | transplant |
| Immune cells | |
| Human CD4+ T-cells | Generation of immune cells/tissue |
| | for transplant |
| Human CD8+ T-cells | Generation of immune cells/tissue |
| | for transplant |
| Human NK cells | Generation of immune cells/tissue |
| | for transplant |
| Human monocytes | Generation of immune cells/tissue |
| | for transplant |
| Human macrophages | Generation of immune cells/tissue |
| | for transplant |
| Human dendritic cells | Generation of immune cells/tissue |
| | for transplant |
| Human B-cells | Generation of immune cells/tissue |
| | for transplant |
| Nose | |
| Goblet cells (mucus | Generation of cells/tissue for |
| secreting cells of the | sinus tissue repair |
| nose) | |
| Pseudostriated ciliated | Generation of cells/tissue for |
| columnar cells (located | sinus tissue repair/replacement |
| below olfactory region in | |
| the nose) | |
| Pseudostratified ciliated | Generation of cells/tissue for |
| epithelium (cells that line | sinus tissue repair/replacement |
| the nasopharangeal tubes) | |
| Trachea | |
| Stratified Epithelial cells | Generation of cells/tissue for |
| (cells that line and | trachea repair/replacement |
| structure the trachea) | |
| Ciliated Columnar cells | Generation of cells/tissue for |
| (cells that line and | trachea repair/replacement |
| structure the trachea) | |
| Goblet cells (cells that | Generation of cells/tissue for |
| line and structure the | trachea repair/replacement |
| trachea) | |
| Basal cells (cells that | Generation of cells/tissue for |
| line and structure the | trachea repair/replacement |
| trachea) | |
| Oesophagus | |
| Cricopharyngeus muscle | Generation of cells/tissue for |
| cells | oesophagus repair/replacement |
| Reproduction | |
| Female primary follicles | Generation of natural fertility |
| Male spermatogonium | Generation of natural fertility |

In terms of using the cells for tissue replacement therapy or augmentation therapy, at least one population of cells may come from the subject to be treated or from a histocompatibility matched subject (i.e. an HLA-matched subject). Furthermore, at birth, subjects may store cells or tissue for the use of the subject (or other suitable subject) later in life. Such tissue would include placenta tissue, umbilical chord tissue, foreskin, blood or other uteric tissue associated with a fetus.

Additional use is described in the copending application.

Persons skilled in the art will appreciate that numerous variations and modifications will become apparent. All such variations and modifications which become apparent to persons skilled in the art, should be considered to fall within the spirit and scope that the invention broadly appearing before described.

Accordingly, while the above description has focused on cell fusion, it will be appreciated that the techniques may generally be applied to any cells, vectors, particles, molecules, liposomes, and other such vesicles.

1) A method of fusing first and second cells, the method including:
a) Selecting at least one pair of a first and a second cell;
b) Individually positioning each pair of cells such that the respective first and second cells of each cell pair are between two electrodes in a fluid filled fusing container, each cell pair being separated from each electrode and from each other cell pair; and,
c) Applying a current having a predetermined waveform to the electrodes to generate a predetermined fusion pulse thereby causing the respective first and second cell of at least one pair of cells to fuse.

2) A method according to claim 1, the cells being held in suspension between the electrodes.

3) A method according to claim 1, the method including generating a DEP field, the DEP field being adapted to urge the cells towards each other.

4) (canceled)

4) A method according to claim 3, the method including applying a current representing the DEP field to a pair of second electrodes.

5) A method according to claim 3, the method including:
a) Applying a DEP current to the pair of second electrodes;
b) Positioning the first cell in the fusing container, the alternating field acting to attract the first cell towards one of the second pair of electrodes; and,
c) Positioning the second cell in the fusing container, the alternating field acting to attract the second cell towards the first cell.

6) A method according to claim 5, at least one of the first and second cells being positioned in contact with at least one of the second pair of electrodes.

7) A method according to claim 1, the method including:
a) Selecting the first and second cells using a pipette:
b) Using the pipette to position the first cell in the fusing container;
c) Using the pipette to position the second cell in the fusing container adjacent the first cell; and,
d) Positioning the electrodes such that the first and second cells are located substantially between the electrodes.

9)-11) (canceled)

8) A method according to claim 7, the method of using the pipette to position the second cell adjacent the first cell including causing the controller to:
a) Move the pipette such that the port is adjacent the first cell in the fusing container;
b) Cause the pipette to expel fluid through the port, thereby expelling the second into the fluid in the fusing container;
c) Move the pipette such that the port is as close as possible to both the first and second cells;
d) Cause the pipette to draw in fluid through the port, thereby drawing in the first and second cells and the surrounding fluid;
e) Cause the pipette to expelling the first and second cells into the fluid in the fusing container; and,
f) Repeat steps (c) to (e) until the first and second cells are within a predetermined distance.

9) A method according to claim 1, the electrodes being coupled to an electrode drive system adapted to move the electrodes with respect to the fusing containers, the method including using a controller coupled to the electrode drive system to position the electrodes in the fusing chamber.

10) A method according to claim 1, the electrodes being coupled to a signal generator, the method of applying the current to the electrodes including causing the signal generator to apply a predetermined current to the electrodes.

11) A method according to claim 10, the first and second cells having a respective cell type, the method including using a controller coupled to a signal generator to select the current in accordance with the cell types of the first and second cells.

16)-17) (canceled)

12) Apparatus for fusing first and second cells, the apparatus including:
a) A fluid filled fusing container;
b) At least two electrodes adapted to be positioned in the fusing container in use;
c) A selector for:
i) Selecting a first cell from a group of first cells held in a respective container; and,
ii) Selecting a second cell from a group of second cells held in a respective container;
iii) Individually positioning the respective first and second cells in the fusing container between the electrodes, so that the cell pair is separated from each electrode and each other pair of cells; and,
d) A signal generator coupled to the electrodes, the signal generator being adapted to cause a field having a predetermined waveform to be generated between the electrodes, thereby causing the respective first and second cells of at least one pair of cells to fuse.

13) Apparatus according to claim 12, the selector being a pipette.

14) Apparatus according to claim 13, the apparatus further including:
a) A drive system adapted to move the pipette with respect to the first, second and fusing containers; and,
b) An actuator adapted to cause the pipette to expel or draw in fluid through a port.

15) Apparatus according to claim 12, the electrodes being coupled to the fusing container.

16) Apparatus according to claim 12, the apparatus further including an electrode drive system adapted to move the electrodes with respect to the fusing containers.

17) Apparatus according to claim 12, the current waveform including:
a) a fusion pulse, the signal generator being adapted to apply the fusion pulse to the electrodes to generate an electric field pulse thereby causing the cells to fuse, and
b) a DEP current, the signal generator being adapted to apply the DEP current to the electrodes to generate a DEP field thereby urging the cells towards each other.

24) (canceled)

18) Apparatus according to claim 12, the apparatus including a pair of second electrodes, the pair of second electrodes being coupled to a second signal generator, the second signal generator being adapted to generate a DEP current, the DEP current being applied to the pair of second electrodes to generate a DEP field thereby urging the cells towards each other.

19) Apparatus according to claim 18, the pair of second electrodes being provided on the fusing container surface.

20) Apparatus according to claim 13, the apparatus further including a controller adapted to control the fusing of the cells by controlling operation of at least one of:
a) The pipette;
b) The electrodes; and,
c) The signal generator.

21) Apparatus according to claim 20, the controller including a processor coupled to at least one of:
a) The drive system and the actuator, the processor being adapted to move and actuate the pipette;
b) The electrode drive system, the processor being adapted to move the electrodes; and,
c) The signal generator, the processor being adapted to cause the signal generator to generate an electrical current having the predetermined waveform.

22) Apparatus according to claim 21, the controller including a detector adapted to detect the position of cells within the containers, the processor being responsive to the detector to move at least one of the electrodes and the pipette in response to the position of detected cells.

30) (canceled)

23) Apparatus according to claim 21, the processor being coupled to a store for storing waveform data representing a number of different predetermined waveforms, the processor being adapted to select one of the number of predetermined waveforms in response to the input commands received from a user.

32)-34) (canceled)

24) A controller for controlling apparatus for fusing first and second cells, the apparatus including:
a) A fluid filled fusing container;
b) At least two electrodes;
c) A selector;
d) A signal generator coupled to the electrodes;
Wherein, in use, the controller is adapted to cause the cells to fuse by:
i) Causing the selector to:
(1) Select a first cell from a group of first cells held in a respective container; and,
(2) Select a second cell from a group of second cells held in a respective container; and,
(3) Individually position the respective first and second cells in the fusing container between the electrodes, the first and second cells being held in suspension, so that the cell pair is being separated from each electrode and each other pair of cells; and,
ii) Causing the signal generator apply a field having a predetermined waveform to the electrodes, thereby causing the respective first and second cells of at least one pair of cells to fuse.

25) A controller according to claim 24, the controller being further adapted to position the electrodes in the fusing container.

26) A controller according to claim 25, the controller including processor coupled to at least one of:
a) A drive system adapted to move a pipette with respect to the first, second and fusing containers;
b) An actuator adapted to cause a pipette to expel or draw in fluid through a port;
c) An electrode drive system adapted to move the electrodes with respect to the fusing containers; and,
d) The signal generator.

27) A controller according to claim 26, the controller including a detector adapted to detect the position of cells within the containers, the processor being responsive to the detector to move at least one of the electrodes and the pipette in response to the position of detected cells.

39) (canceled)

28) A controller according to claim 26, the processor being coupled to a store for storing waveform data representing a number of different predetermined waveforms, the processor being adapted to select one of the number of predetermined waveforms in response to the input commands received from the user.

29) A controller according to claim 26, the processor being adapted to move at least one of the electrodes and the pipette in response to the input commands received from a user.

30) A controller according to claim 24, the current waveform including at least one of:
a) a fusion pulse, the controller being adapted to cause the signal generator to apply the fusion pulse to the electrodes to generate an electric field pulse thereby causing the cells to fuse; and
b) a DEP current, the controller being adapted to cause the signal generator to apply the DEP current to the electrodes to generate a DEP field thereby urging the cells towards each other.

31) A controller according to any one of the claim 24, the apparatus including a pair of second electrodes, the pair of second electrodes being coupled to a second signal generator, the controller being adapted to cause the second signal generator to generate a DEP current, the DEP current being applied to the pair of second electrodes to generate a DEP field thereby urging the cells towards each other.

32) A controller according to claim 24, the controller being adapted for use with apparatus including:
a) A fluid filled fusing container;
b) At least two electrodes adapted to be positioned in the fusing container in use;
c) A selector for:
i. Selecting a first cell from a group of first cells held in a respective container; and
ii. Selecting a second cell from a group of second cells held in a respective container;
iii. Individually positioning the respective first and second cells in the fusing container between the electrodes so that the cell pair is separated from each electrode and each other pair of cells; and,
d) A signal generator coupled to the electrodes, the signal generator being adapted to cause a field having a predetermined waveform to be generated between the electrodes, thereby causing the respective first and second cells of at least one pair of cells to fuse.

46)-50) (canceled)

33) A pipette system for manipulating particles, the pipette system including:
a) A nozzle for containing fluid in use, the nozzle including a port;
b) An actuator coupled to the nozzle, the actuator being adapted to draw in and/or expel fluid through the port; and,
c) An electrode coupled to the nozzle adjacent to the port, the electrode being adapted to cooperate with a second electrode to allow an electric field to be applied to coupled to one or more particles positioned adjacent to the port.

34) A pipette system according to claim 33, the electrode being formed a conductive tube.

35) A pipette system according to claim 34, the electrode being formed from a stainless steel tube having a diameter of approximately 10 mm.

36) A pipette system according to claim 33, the pipette system including a drive system adapted to move the pipette system to be with respect to a fluid filled container to thereby allow particles to be positioned in or removed from fluid in the container.

37) A pipette system according to claim 36, the pipette system including a signal generator coupled to the electrode for generating a predetermined electric field between the electrode and a second electrode positioned in the container.

38) A pipette system according to claim 37, the pipette system including a controller adapted to control the drive system, the actuator and the signal generator to thereby apply an electric field to a particle by:
a) Positioning the particle in the container adjacent the second electrode using the pipette;
b) Positioning the pipette port adjacent the particle in the container; and,
c) Activating the signal generator.

39) A pipette system according to claim 38, the controller being adapted to fuse cells, by:
a) Positioning a first cell in the container adjacent the second electrode using the pipette;
b) Positioning a second cell in the container adjacent the first cell using the pipette;
c) Positioning the pipette port adjacent the first and second cells, such that first and second cells are substantially between the electrodes; and,
d) Activating the signal generator to cause a predetermined field sequence to be applied to the cells, thereby causing the cells to fuse.

40) A pipette system according to claim 39, the pipette system further including:
a) A radiation source; and,
b) waveguide having a first end coupled to the radiation source and a second end coupled to the nozzle adjacent the port to thereby allow radiation from the radiation source to impinge on particles positioned adjacent to the port in use.

41) A pipette system according to claim 40, the pipette system including a detector, the detector being adapted to detect radiation emitted by the particle.

42) A pipette system according to claim 41, the detector being coupled to the first end of the waveguide, to thereby detect radiation emitted from the particle.

43) A pipette system according to claim 40, the radiation source being a laser.

44) A pipette system according to claim 40, the waveguide being a fibre optic cable.

45) A pipette system according to claim 40, the waveguide being formed from the nozzle, the nozzle including a shaped portion to allow the radiation from the radiation source to enter the nozzle and pass along at least a portion of the nozzle, the radiation being emitted from the nozzle through the port.

46) A pipette system according to claim 40, the pipette system including a controller adapted to perform at least one of:
a) activating the actuator to thereby cause fluid to be drawn in and/or expelled through the port; and,
b) Activating the radiation source, to thereby expose a particle to radiation.

47) A pipette system according to claim 46, the drive system being coupled to a controller, the controller being adapted to recover particles having predetermined properties from the container by:
a) Positioning the pipette system such that the port is adjacent to a particle;
b) Activating the radiation source to thereby expose the particle to radiation;
c) Detect any radiation emitted by the particle;
d) Determine if the particle has the predetermined properties in accordance with the detected radiation; and,
e) In accordance with a successful comparison, activate the actuator to thereby draw fluid into the nozzle through the port, thereby recovering the particle.

48) A pipette system according to claim 47, the actuator including:
a) A fluid reservoir;
b) A flexible tube coupling the nozzle to the fluid reservoir;
c) An arm positioned so as to partially compress the tube;
d) An actuator drive system adapted to move the arm so as to perform at least one of:
i. Further compressing the tube to thereby expel fluid from the port; and,
ii. Decompressing the tube to thereby draw fluid in through the port.

49) A pipette system according to claim 48, the actuator drive system including:
a) A first actuator drive for moving the arm with respect to the tube and/or a bladder; and,
b) A second actuator drive formed from an arm end portion, the arm end portion being in contact with the tube in use, the second actuator drive being adapted to cause the arm end portion to expand or contract.

50) A pipette system according to claim 48, the pipette system including a controller coupled to the actuator drive system, the controller being adapted to operate the actuator drive system to thereby draw fluid in or expel fluid through the port.

51) A pipette system according to claim 50, the drive system being coupled to the controller, the controller being adapted to recover particles from the fluid by:
a) Positioning the pipette system such that the port is adjacent to a particle; and,
b) Activate the actuator drive system to thereby draw fluid into the nozzle through the port, thereby recovering the particle.

52) A pipette system according to claim 48, the tube being formed from silicon tubing.

71) (canceled)
